SEQUENCE LISTING (22 sequences)

SEQ ID NO: 1 - DNA, 848 bp, Homo sapiens
  CDS (5)...(568)
gaaaatggcgctggccatgctggtcttggtggtttcgccgtggtctgcggcccggggagtgcttcgaaactactgggagcgactgctacggaagcttccgcagagccggccgggctttcccagtcctccgtggggaccagcattagcagtacagggcccagccatgtttacagagccagcaaatgataccagtggaagtaaagagaattccagccttttggacagtatcttttggatggcagctcccaaaaatagacgcaccattgaagttaaccggtgtaggagaagaaatccgcagaagcttattaaagttaagaacaacatagacgtttgtcctgaatgtggtcacctgaaacagaaacatgtcctttgtgcctactgctatgaaaaggtgtgcaaggagactgcagaaatcagacgacagatagggaagcaagaagggggcccttttaaggctcccaccatagagactgtggtgctgtacacaggagagacaccgtctgaacaagatcagggcaagaggatcattgaacgagacagaaagcgaccatcctggttcacccagaattgacaccaaagatgttaaaaggataacttcacagtaaatcatttctcctgaaatagaggaagattctttacgttgttgtgcttgtttttaaatcatcagtatagtttaacacattctttctaagcagttttgtgtgggataatttgaagaatatattatgagtaaactccgaaaattttgtttatccaaaggctcaatggattatgtttctattatatacaaggttttaagtaaacataaaatttccagaacaaaaataaaaaatttaaaattcatagca

SEQ ID NO: 2 - protein, 188 aa, Homo sapiens [sequence omitted]

SEQ ID NO: 3 - DNA, 564 bp, Artificial Sequence
  Degenerate polynucleotide sequence of zpep14
atggcnytngcnatgytngtnytngtngtnwsnccntggwsngcngcnmgnggngtnytnmgnaaytaytgggarmgnytnytnmgnaarytnccncarwsnmgnccnggnttyccnwsnccnccntggggnccngcnytngcngtncarggnccngcnatgttyacngarccngcnaaygayacnwsnggnwsnaargaraaywsnwsnytnytngaywsnathttytggatggcngcnccnaaraaymgnmgnacnathgargtnaaymgntgymgnmgnmgnaayccncaraarytnathaargtnaaraayaayathgaygtntgyccngartgyggncayytnaarcaraarcaygtnytntgygcntaytgytaygaraargtntgyaargaracngcngarathmgnmgncarathggnaarcargarggnggnccnttyaargcnccnacnathgaracngtngtnytntayacnggngaracnccnwsngarcargaycarggnaarmgnathathgarmgngaymgnaarmgnccnwsntggttyacncaraay

SEQ ID NO: 4 - DNA, 23 bp, Artificial Sequence
  Oligonucleotide primer ZC18791
gggaaaatggcgctggccatgct

SEQ ID NO: 5 - DNA, 23 bp, Artificial Sequence
  Oligonucleotide primer ZC18792
aacatggctgggccctgtactgc

SEQ ID NO: 6 - DNA, 22 bp, Artificial Sequence
  Oligonucleotide primer ZC20516
ggtgtcaattctgggtgaacca

SEQ ID NO: 7 - DNA, 20 bp, Artificial Sequence
  Oligonucleotide primer ZC694
taatacgactcactataggg

SEQ ID NO: 8 - DNA, 25 bp, Artificial Sequence
  Oligonucleotide primer ZC6768
gcaattaaccctcactaaagggaac

SEQ ID NO: 9 - DNA, 20 bp, Artificial Sequence
  Oligonucleotide primer ZC7710
gttgtgtggaattgtgagcg

SEQ ID NO: 10 - DNA, 16 bp, Artificial Sequence
  Oligonucleotide primer ZC3424
aacagctatgaccatg

SEQ ID NO: 11 - DNA, 22 bp, Artificial Sequence
  Oligonucleotide primer ZC18143
catgtcctttgtgcctactgct

SEQ ID NO: 12 - DNA, 21 bp, Artificial Sequence
  Oligonucleotide primer ZC18144
ggtgtcaattctgggtgacca

SEQ ID NO: 13 - DNA, 18 bp, Artificial Sequence
  Oligonucleotide primer ZC21355
cagccttttggacagtat

SEQ ID NO: 14 - DNA, 18 bp, Artificial Sequence
  Oligonucleotide primer ZC21356
gcttctgcggatttcttc

SEQ ID NO: 15 - DNA, 19 bp, Artificial Sequence
  Oligonucleotide primerZC20672
agcaccatagtctcaacgg

SEQ ID NO: 16 - DNA, 21 bp, Artificial Sequence
  Oligonucleotide primer ZC20622
acctgaagcagaaacatgtcc

SEQ ID NO: 17 - DNA, 794 bp, Mus musculus
  CDS (48)...(611)
ggatccatgtgcgctcagcccgcgtcccgctctgtcttgagaagatcatggctccttcgttgctgctgctttcactaccatggccggtgcgtcccggaccgctccagaggtgctgggagctgctacaacggcaactgcagcagagctggagtcgctttgtcagtcctccgtgggcaccagcattagccgtccagagaccatccatcctcacagagctagcacatgatacttgtgaaaataaagagaattccagctttttagatagtatcttttggatggcagctcccaaaaacagacgcaccatcgaagttaaccgatgtaggagaagaaaccctcaaaagcttattaaaattaagaacaatatagacatttgccctgaatgtggtcacctgaagcagaaacatgtcctttgtggatattgctatgagaaagtacgccaggagacgacaaaaatcagacaacaaataggggctcaagaaggaggtcctttcagagctccttccgttgagactatggtgctgtacacaggagagaaaccatcagaaaaggatcagggcaagaggattgttgaaagaaacataaagaggccatcttggttcacccagaattgaactaaaaaatgttaaaaagataatttcatagttaggcacttatttaaaatcaggagtcttcattttcatgttgtgcctgttttcaaattatcaatgtattttaaaatggattttgttagttgttacttttaaatgaggtaatttgaagactgtatcatgaataaactcagcatatttatgtaa

SEQ ID NO: 18 - protein, 187 aa, Mus musculus
MAPSLLLLSLPWPVRPGPLQRCWELLQRQLQQSWSRFVSPPWAPALAVQRPSILTELAHDTCENKENSSFLDSIFWMAAPKNRRTIEVNRCRRRNPQKLIKIKNNIDICPECGHLKQKHVLCGYCYEKVRQETTKIRQQIGAQEGGPFRAPSVETMVLYTGEKPSEKDQGKRIVERNIKRPSWFTQN

SEQ ID NO: 19 - DNA, 32 bp, Artificial Sequence
  Oligonucleotide primer ZC20886
gtatacggccggccaccatggcgctggccatg

SEQ ID NO: 20 - DNA, 32 bp, Artificial Sequence
  Oligonucleotide primer ZC20887
cgcgcgggcgcgcctcaattctgggtgaacca

SEQ ID NO: 21 - protein, 32 aa, Homo sapiens
  AMIDATION (32)...(32)
QIGKQEGGPFKAPTIETVVLYTGETPSEQDQG

SEQ ID NO: 22 - protein, 15 aa, Homo sapiens
IIERDRKRPSWFTQN

(19) United States
(12) Patent Application Publication
(10) Pub. No.: US 2003/0083478 A1
(43) Pub. Date: May 1, 2003

(54) Prostate, testis and uterine polypeptide zpep14

(76) Inventors: James L. Holloway, Seattle, WA (US); Paul D. Bishop, Fall City, WA (US); Zeren Gao, Redmond, WA (US)

Correspondence Address: Jennifer K Johnson JD, ZymoGenetics Incorporation, 1201 Eastlake Avenue East, Seattle, WA, 98102, US

(21) Appl. No.: 09/497,549

(22) Filed: Feb. 3, 2000

Related U.S. Application Data
(60) Provisional application No. 60/119,173, filed on Feb. 8, 1999.
(60) Provisional application No. 60/151,035, filed on Aug. 27, 1999.

(51) Int. Cl. G01N 33/53; C07H 21/02; C07H 21/04; C12P 21/06; C07K 2/00; C07K 4/00; C07K 5/00; C07K 7/00; C07K 14/00; C07K 16/00; C07K 17/00; A61K 38/00

(52) U.S. Cl. 536/23.1; 435/7.1; 530/300

(57) ABSTRACT

The present invention relates to polynucleotide and polypeptide molecules for zpep14, a novel secreted protein. The polynucleotides encoding zpep14, may, for example, be used to identify a region of the genome associated with human disease states. The present invention also includes methods for producing the protein, uses therefor and antibodies thereto.

### REFERENCE TO RELATED APPLICATIONS

This application is related to Provisional Application 60/119,173, filed on Feb. 8, 1999. This application is also related to Provisional Application 60/151,035, filed on Aug. 27, 1999. Under 35 U.S.C. §119(e)(1), this application claims benefit of said Provisional Applications.

### BACKGROUND OF THE INVENTION

Mammalian neurokinins (also referred to as tackykinins) are small peptides that appear to be involved in numerous physiological functions. Such neurokinins include substance P (SP), an 11 amino acid polypeptide; neurokinin A (NKA, also referred to as neuromedin L and substance K), a 10 amino acid polypeptide; and neurokinin B (NKB, also known as neuromedin K, neuromedin B and neurokinin K), a ten amino acid polypeptide. Three mammalian neurokinin receptors have been identified, each with a characteristic neurokinin binding preference pattern. See, for example, Maggi, General Pharmacology (United Kingdom) 26(5): 911-44, 1995; Huber et al., Eur. J. Pharmacol. (Netherlands) 239(1-3): 103-9, 1993; and, Maggi et al., Regulatory Peptides 53: 259-74, 1994.

Mammalian neurokinins are generally expressed in the form of precursor proteins. Cleavage of precursor proteins releases active neurokinins. For example, a bovine NKB precursor protein is described in Kotani et al., Proc. Natl. Acad. Sci. (USA) 83: 7074-8, 1986. The deduced amino acid sequence of the disclosed bovine NKB precursor is 126 amino acid residues long with a putative signal sequence at the 5′ end thereof.

Neurokinins have been implicated in a number of physiological processes. Such processes include neurotransmission/neuromodulation in the nervous system and peripheral tissues, smooth muscle contraction (e.g., in respiratory, gastrointestinal and urinary tissue), growth/proliferation (e.g., small cell carcinoma), hormone secretion (e.g., pancreas, pituitary gland and gastrin-secreting cells), inhibition of gastric emptying, modulation of neutrophil function, blood pressure regulation and the like. See, for example, Kotani et al. (referenced above); Belloli et al., J. Vet. Pharmacol. Therap. 17: 379-83, 1994; Battey et al., Journal of the National Cancer Institute Monographs 13: 141-4, 1992; Henriksen et al., J. of Receptor & Signal Transduction Research 15(1-4): 529-41, 1995; Dobrzanski et al., Regulatory Peptides 45: 341-52, 1993; Varga et al., Eur. J. Pharmacology 286: 109-112, 1995; Wozniak et al., Immunology 78: 629-34, 1993; Munekata, Comp. Biochem. Physiol. 98C(1): 171-9, 1991; and Ding et al., J. Comparative Neurology 364: 290-310, 1996.

Neurokinins are generally expressed as precursor molecules encompassing the active polypeptides. Evidence exists that precursor polypeptides can be more effective upon administration than active protein alone. Polypeptide precursors of neurokinins are therefore sought for the study of neurokinin-related physiological processes. Moreover, novel polypeptides and polypeptide precursors with neurokinin-like functions are sought. The present invention provides such polypeptides for these and other uses that should be apparent to those skilled in the art from the teachings herein.

### SUMMARY OF THE INVENTION

The present invention addresses this need by providing a novel polypeptide and related compositions and methods.

The present invention provides an isolated polynucleotide encoding a zpep14 polypeptide comprising a sequence of amino acid residues that is at least 90% identical to an amino acid sequence selected from the group consisting of: (a) the amino acid sequence as shown in SEQ ID NO:2 from amino acid number 17 (Arg) to amino acid number 92 (Cys); (b) the amino acid sequence as shown in SEQ ID NO:2 from amino acid number 96 (Asn) to amino acid number 137 (Ile); (c) the amino acid sequence as shown in SEQ ID NO:2 from amino acid number 140 (Gln) to amino acid number 171 (Gly); (d) the amino acid sequence as shown in SEQ ID NO:2 from amino acid number 17 (Arg) to amino acid number 137 (Ile); (e) the amino acid sequence as shown in SEQ ID NO:2 from amino acid number 17 (Arg) to amino acid number 171 (Gly); (f) the amino acid sequence as shown in SEQ ID NO:2 from amino acid number 96 (Asn) to amino acid number 171 (Gly); (g) the amino acid sequence as shown in SEQ ID NO:2 from amino acid number 174 (Ile) to amino acid number 188 (Asn); (h) the amino acid sequence as shown in SEQ ID NO:2 from amino acid number 140 (Gln) to amino acid number 188 (Asn); (i) the amino acid sequence as shown in SEQ ID NO:2 from amino acid number 96 (Asn) to amino acid number 188 (Asn); (j) the amino acid sequence as shown in SEQ ID NO:2 from amino acid number 17 (Arg) to amino acid number 188 (Asn); and (k) the amino acid sequence as shown in SEQ ID NO:2 from amino acid number 1 (Met) to amino acid number 188 (Asn), wherein the amino acid percent identity is determined using a FASTA program with ktup=1, gap opening penalty=10, gap extension penalty=1, and substitution matrix=BLOSUM62, with other parameters set as default. Within one embodiment the isolated polynucleotide disclosed above is selected from the group consisting of: (a) a polynucleotide sequence as shown in SEQ ID NO:1 from nucleotide 53 to nucleotide 280; (b) a polynucleotide sequence as shown in SEQ ID NO:1 from nucleotide 290 to nucleotide 415; (c) a polynucleotide sequence as shown in SEQ ID NO:1 from nucleotide 422 to nucleotide 517; (d) a polynucleotide sequence as shown in SEQ ID NO:1 from nucleotide 53 to nucleotide 415 ; (e) a polynucleotide sequence as shown in SEQ ID NO:1 from nucleotide 53 to nucleotide 517; (f) a polynucleotide sequence as shown in SEQ ID NO:1 from nucleotide 290 to nucleotide 517; (g) a polynucleotide sequence as shown in SEQ ID NO:1 from nucleotide 524 to nucleotide 568; (h) a polynucleotide sequence as shown in SEQ ID NO:1 from nucleotide 422 to nucleotide 568; (i) a polynucleotide sequence as shown in SEQ ID NO:1 from nucleotide 290 to nucleotide 568; (j) a polynucleotide sequence as shown in SEQ ID NO:1 from nucleotide 53 to nucleotide 568; (k) a polynucleotide sequence as shown in SEQ ID NO:1 from nucleotide 5 to nucleotide 568; and (l) a polynucleotide sequence complementary to (a) through (k). Within another embodiment the isolated polynucleotide disclosed above comprises nucleotide 1 to nucleotide 564 of SEQ ID NO:3. Within another embodiment the isolated polynucleotide disclosed above comprises a sequence of amino acid residues selected from the group consisting of: (a) the amino acid sequence as shown in SEQ ID NO:2 from amino acid number 17 (Arg) to amino acid number 92 (Cys); (b) the amino acid sequence as shown in SEQ ID NO:2 from amino acid number 96 (Asn) to amino acid number 137 (Ile); (c) the amino acid sequence as shown in SEQ ID NO:2 from amino acid number 140 (Gln) to amino acid number 171 (Gly); (d) the amino acid sequence as shown in SEQ ID NO:2 from amino acid number 17 (Arg) to amino acid number 137 (Ile); (e) the amino acid sequence as shown in SEQ ID NO:2 from amino acid number 17 (Arg) to amino acid number 171 (Gly); (f) the amino acid sequence as shown in SEQ ID NO:2 from amino acid number 96 (Asn) to amino acid number 171 (Gly); (g) the amino acid sequence as shown in SEQ ID NO:2 from amino acid number 174 (Ile) to amino acid number 188 (Asn); (h) the amino acid sequence as shown in SEQ ID NO:2 from amino acid number 140 (Gln) to amino acid number 188 (Asn); (i) the amino acid sequence as shown in SEQ ID NO:2 from amino acid number 96 (Asn) to amino acid number 188 (Asn); (j) the amino acid sequence as shown in SEQ ID NO:2 from amino acid number 17 (Arg) to amino acid number 188 (Asn); and (k) the amino acid sequence as shown in SEQ ID NO:2 from amino acid number 1 (Met) to amino acid number 188 (Asn). Within another embodiment the isolated polynucleotide disclosed above consists of a sequence of amino acid residues as shown in SEQ ID NO:2 from amino acid number 96 (Asn) to amino acid number 137 (Ile).

Within a second aspect the present invention provides an expression vector comprising the following operably linked elements: a transcription promoter; a DNA segment encoding a zpep14 polypeptide that is at least 90% identical to an amino acid sequence as shown in SEQ ID NO:2 from amino acid number 17 (Arg) to amino acid number 188 (Asn); and a transcription terminator. In one embodiment the expression vector disclosed above further comprises a secretory signal sequence operably linked to the DNA segment.

Within a third aspect the present invention provides a cultured cell into which has been introduced an expression vector as disclosed above, wherein the cell expresses a polypeptide encoded by the DNA segment.

Within a fourth aspect the present invention provides a DNA construct encoding a fusion protein, the DNA construct comprising: a first DNA segment encoding a polypeptide that is at least 90% identical to a sequence of amino acid residues selected from the group consisting of: (a) the amino acid sequence of SEQ ID NO: 2 from residue number 1 (Met) to residue number 16 (Ala)); and at least one other DNA segment encoding an additional polypeptide, wherein the first and other DNA segments are connected in-frame; and encode the fusion protein.

Within another aspect the present invention provides a fusion protein produced by a method comprising:culturing a host cell into which has been introduced a vector comprising the following operably linked elements: (a) a transcriptional promoter; (b) a DNA construct encoding a fusion protein as disclosed above; and (c) a transcriptional terminator; and recovering the protein encoded by the DNA segment.

Within another aspect the present invention provides an isolated polypeptide comprising a sequence of amino acid residues that is at least 90% identical to an amino acid sequence selected from the group consisting of: (a) the amino acid sequence as shown in SEQ ID NO:2 from amino acid number 17 (Arg) to amino acid number 92 (Cys); (b) the amino acid sequence as shown in SEQ ID NO:2 from amino acid number 96 (Asn) to amino acid number 137 (Ile); (c) the amino acid sequence as shown in SEQ ID NO:2 from amino acid number 140 (Gln) to amino acid number 171 (Gly); (d) the amino acid sequence as shown in SEQ ID NO:2 from amino acid number 17 (Arg) to amino acid number 137 (Ile); (e) the amino acid sequence as shown in SEQ ID NO:2 from amino acid number 17 (Arg) to amino acid number 171 (Gly); (f) the amino acid sequence as shown in SEQ ID NO:2 from amino acid number 96 (Asn) to amino acid number 171 (Gly); (g) the amino acid sequence as shown in SEQ ID NO:2 from amino acid number 174 (Ile) to amino acid number 188 (Asn); (h) the amino acid sequence as shown in SEQ ID NO:2 from amino acid number 140 (Gln) to amino acid number 188 (Asn); (i) the amino acid sequence as shown in SEQ ID NO:2 from amino acid number 96 (Asn) to amino acid number 188 (Asn); (j) the amino acid sequence as shown in SEQ ID NO:2 from amino acid number 17 (Arg) to amino acid number 188 (Asn); and (k) the amino acid sequence as shown in SEQ ID NO:2 from amino acid number 1 (Met) to amino acid number 188 (Asn), wherein the amino acid percent identity is determined using a FASTA program with ktup=1, gap opening penalty=10, gap extension penalty=1, and substitution matrix=BLOSUM62, with other parameters set as default. In one embodiment, the isolated polypeptide disclosed above comprises a sequence of amino acid residues that is selected from the group consisting of: (a) the amino acid sequence as shown in SEQ ID NO:2 from amino acid number 17 (Arg) to amino acid number 92 (Cys); (b) the amino acid sequence as shown in SEQ ID NO:2 from amino acid number 96 (Asn) to amino acid number 137 (Ile); (c) the amino acid sequence as shown in SEQ ID NO:2 from amino acid number 140 (Gln) to amino acid number 171 (Gly); (d) the amino acid sequence as shown in SEQ ID NO:2 from amino acid number 17 (Arg) to amino acid number 137 (Ile); (e) the amino acid sequence as shown in SEQ ID NO:2 from amino acid number 17 (Arg) to amino acid number 171 (Gly); (f) the amino acid sequence as shown in SEQ ID NO:2 from amino acid number 96 (Asn) to amino acid number 171 (Gly); (g) the amino acid sequence as shown in SEQ ID NO:2 from amino acid number 174 (Ile) to amino acid number 188 (Asn); (h) the amino acid sequence as shown in SEQ ID NO:2 from amino acid number 140 (Gln) to amino acid number 188 (Asn); (i) the amino acid sequence as shown in SEQ ID NO:2 from amino acid number 96 (Asn) to amino acid number 188 (Asn); (j) the amino acid sequence as shown in SEQ ID NO:2 from amino acid number 17 (Arg) to amino acid number 188 (Asn); and (k) the amino acid sequence as shown in SEQ ID NO:2 from amino acid number 1 (Met) to amino acid number 188 (Asn). In another embodiment, the isolated polypeptide disclosed above is as shown in SEQ ID NO:2 from amino acid number 96 (Asn) to amino acid number 137 (Ile).

Within another aspect the present invention provides a method of producing a zpep14 polypeptide comprising: culturing a cell as disclosed above; and isolating the zpep14 polypeptide produced by the cell.

Within another aspect the present invention provides a method of detecting, in a test sample, the presence of a modulator of zpep14 protein activity, comprising:transfecting a zpep14-responsive cell, with a reporter gene construct that is responsive to a zpep14-stimulated cellular pathway; and producing a zpep14 polypeptide by the method as disclosed above; and adding the zpep14 polypeptide to the cell, in the presence and absence of a test sample; and comparing levels of response to the zpep14 polypeptide, in the presence and absence of the test sample, by a biological or biochemical assay; and determining from the comparison, the presence of the modulator of zpep14 activity in the test sample.

Within another aspect the present invention provides a method of producing an antibody to zpep14 polypeptide comprising the following steps in order: inoculating an animal with a polypeptide selected from the group consisting of: (a) a polypeptide consisting of 9 to 172 amino acids, wherein the polypeptide is at least 90% identical to a contiguous sequence of amino acids in SEQ ID NO:2 from amino acid number 17 (Arg) to amino acid number 188 (Asn); (b) a polypeptide consisting of the amino acid sequence of SEQ ID NO: 2 from amino acid number 17 (Arg) to amino acid number 188 (Asn); (c) a polypeptide as disclosed above; (d) a polypeptide consisting of amino acid number 90 (Asn) to amino acid number 95 (Arg) of SEQ ID NO:2; (e) a polypeptide consisting amino acid number 128 (Glu) to amino acid number 133 (Glu) of SEQ ID NO:2; (f) a polypeptide consisting of amino acid number 167 (Glu) to amino acid number 172 (Lys) of SEQ ID NO:2; (g) a polypeptide consisting of amino acid number 175 (Ile) to amino acid number 180 (Lys) of SEQ ID NO:2; and (h) a polypeptide consisting of amino acid number 176 (Glu) to amino acid number 181 (Arg) of SEQ ID NO:2; and wherein the polypeptide elicits an immune response in the animal to produce the antibody; and isolating the antibody from the animal.

Within another aspect the present invention provides an antibody produced by the method as disclosed above, which binds to a zpep14 polypeptide. In one embodiment, the antibody disclosed above is a monoclonal antibody.

Within another aspect the present invention provides an antibody which specifically binds to a polypeptide as disclosed above.

Within another aspect, the present invention provides an isolated polynucleotide encoding a zpep14 polypeptide comprising a sequence of amino acid residues that is at least 90% identical to an amino acid sequence selected from the group consisting of: (a) the amino acid sequence as shown in SEQ ID NO:18 from amino acid number 17 (Gly) to amino acid number 91 (Cys); (b) the amino acid sequence as shown in SEQ ID NO:18 from amino acid number 17 (Gly) to amino acid number 170 (Gly); (c) the amino acid sequence as shown in SEQ ID NO:18 from amino acid number 95 (Asn) to amino acid number 170 (Gly); (d) the amino acid sequence as shown in SEQ ID NO:18 from amino acid number 173 (Ile) to amino acid number 187 (Asn); (e) the amino acid sequence as shown in SEQ ID NO:18 from amino acid number 17 (Gly) to amino acid number 187 (Asn); and (f) the amino acid sequence as shown in SEQ ID NO:18 from amino acid number 1 (Met) to amino acid number 187 (Asn), wherein the amino acid percent identity is determined using a FASTA program with ktup=1, gap opening penalty=10, gap extension penalty=1, and substitution matrix=BLOSUM62, with other parameters set as default. In one embodiment, the isolated polynucleotide disclosed above is selected from the group consisting of: (a) a polynucleotide sequence as shown in SEQ ID NO:17 from nucleotide 96 to nucleotide 320; (b) a polynucleotide sequence as shown in SEQ ID NO:17 from nucleotide 96 to nucleotide 557; (c) a polynucleotide sequence as shown in SEQ ID NO:17 from nucleotide 330 to nucleotide 557; (d) a polynucleotide sequence as shown in SEQ ID NO:17 from nucleotide 564 to nucleotide 608; (e) a polynucleotide sequence as shown in SEQ ID NO:17 from nucleotide 96 to nucleotide 608; (f) a polynucleotide sequence as shown in SEQ ID NO:17 from nucleotide 48 to nucleotide 608; and (g) a polynucleotide sequence complementary to (a) through (k). In another embodiment, the isolated polynucleotide disclosed above encodes a zpep14 polypeptide comprising a sequence of amino acid residues selected from the group consisting of: (a) the amino acid sequence as shown in SEQ ID NO:18 from amino acid number 17 (Gly) to amino acid number 91 (Cys); (b) the amino acid sequence as shown in SEQ ID NO:18 from amino acid number 17 (Gly) to amino acid number 170 (Gly); (c) the amino acid sequence as shown in SEQ ID NO:18 from amino acid number 95 (Asn) to amino acid number 170 (Gly); (d) the amino acid sequence as shown in SEQ ID NO:18 from amino acid number 173 (Ile) to amino acid number 187 (Asn); (e) the amino acid sequence as shown in SEQ ID NO:18 from amino acid number 17 (Gly) to amino acid number 187 (Asn); and (f) the amino acid sequence as shown in SEQ ID NO:18 from amino acid number 1 (Met) to amino acid number 187 (Asn).

Within another aspect, the present invention provides an expression vector comprising the following operably linked elements: a transcription promoter; a DNA segment encoding a zpep14 polypeptide that is at least 90% identical to an amino acid sequence as shown in SEQ ID NO:18 from amino acid number 17 (Gly) to amino acid number 187 (Asn); and a transcription terminator. In one embodiment, the expression vector disclosed above further comprises a secretory signal sequence operably linked to the DNA segment.

Within another aspect, the present invention provides a cultured cell into which has been introduced an expression vector as disclosed above, wherein the cell expresses a polypeptide encoded by the DNA segment.

Within another aspect, the present invention provides an isolated polypeptide comprising a sequence of amino acid residues that is at least 90% identical to an amino acid sequence selected from the group consisting of: (a) the amino acid sequence as shown in SEQ ID NO:18 from amino acid number 17 (Gly) to amino acid number 91 (Cys); (b) the amino acid sequence as shown in SEQ ID NO:18 from amino acid number 17 (Gly) to amino acid number 170 (Gly); (c) the amino acid sequence as shown in SEQ ID NO:18 from amino acid number 95 (Asn) to amino acid number 170 (Gly); (d) the amino acid sequence as shown in SEQ ID NO:18 from amino acid number 173 (Ile) to amino acid number 187 (Asn); (e) the amino acid sequence as shown in SEQ ID NO:18 from amino acid number 17 (Gly) to amino acid number 187 (Asn); and (f) the amino acid sequence as shown in SEQ ID NO:18 from amino acid number 1 (Met) to amino acid number 187 (Asn), wherein the amino acid percent identity is determined using a FASTA program with ktup=1, gap opening penalty=10, gap extension penalty=1, and substitution matrix=BLOSUM62, with other parameters set as default. In one embodiment, the isolated polypeptide disclosed above comprises a sequence of amino acid residues that is selected from the group consisting of: (a) the amino acid sequence as shown in SEQ ID NO:18 from amino acid number 17 (Gly) to amino acid number 91 (Cys); (b) the amino acid sequence as shown in SEQ ID NO:18 from amino acid number 17 (Gly) to amino acid number 170 (Gly); (c) the amino acid sequence as shown in SEQ ID NO:18 from amino acid number 95 (Asn) to amino acid number 170 (Gly); (d) the amino acid sequence as shown in SEQ ID NO:18 from amino acid number 173 (Ile) to amino acid number 187 (Asn); (e) the amino acid sequence as shown in SEQ ID NO:18 from amino acid number 17 (Gly) to amino acid number 187 (Asn); and (f) the amino acid sequence as shown in SEQ ID NO:18 from amino acid number 1 (Met) to amino acid number 187 (Asn).

Within another aspect, the present invention provides a method of producing a zpep14 polypeptide comprising: culturing a cell as disclosed above; and isolating the zpep14 polypeptide produced by the cell.

Within another aspect, the present invention provides a method of producing an antibody to a zpep14 polypeptide comprising the following steps in order:

inoculating an animal with a polypeptide selected from the group consisting of: (a) a polypeptide as disclosed above; (b) a polypeptide consisting of the amino acid sequence of SEQ ID NO:18 from amino acid number 17 (Gly) to 91 (Cys); (c) a polypeptide consisting of the amino acid sequence of SEQ ID NO:18 from amino acid number 17 (Gly) to 170 (Gly); (d) a polypeptide consisting of the amino acid sequence of SEQ ID NO:18 from amino acid number 95 (Asn) to 170 (Gly); (e) a polypeptide consisting of the amino acid sequence of SEQ ID NO:18 from amino acid number 173 (Ile) to 187 (Asn); (f) a polypeptide consisting of the amino acid sequence of SEQ ID NO:18 from amino acid number 17 (Gly) to 187 (Asn); and (g) a polypeptide consisting of a hydrophilic peptide predicted from a murine zpep14 hydrophobicity plot using a Hopp/Woods hydrophilicity profile based on a sliding six-residue window, with buried G, S, and T residues and exposed H, Y, and W residues ignored; and and wherein the polypeptide elicits an immune response in the animal to produce the antibody; and isolating the antibody from the animal.

Within another aspect, the present invention provides an antibody produced by the method as disclosed above, which binds to a zpep14 polypeptide. In one embodiment, the antibody disclosed above is a monoclonal antibody. In another aspect, the present invention provides an antibody which binds to a polypeptide as disclosed above.

These and other aspects of the invention will become evident upon reference to the following detailed description of the invention and attached drawings.

### BRIEF DESCRIPTION OF THE DRAWINGS

FIG. 1 is a hydrophobicity plot of human zpep14, determined from a Hopp/Woods hydrophilicity profile based on a sliding six-residue window, with buried G, S, and T residues and exposed H, Y, and W residues ignored.

FIG. 2 is a multiple alignment of the human zpep14 polypeptide (SEQ ID NO:2), and mouse zpep14 polypeptide (SEQ ID NO:18) of the present invention.

### DETAILED DESCRIPTION OF THE INVENTION

Prior to setting forth the invention in detail, it may be helpful to the understanding thereof to define the following terms:

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” (N-terminal) and “carboxyl-terminal” (C-terminal) are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹.

The term “complements of a polynucleotide molecule” denotes a polynucleotide molecule having a complementary base sequence and reverse orientation as compared to a reference sequence. For example, the sequence 5′ ATGCACGGG 3′ is complementary to 5′ CCCGTGCAT 3′.

The term “contig” denotes a polynucleotide that has a contiguous stretch of identical or complementary sequence to another polynucleotide. Contiguous sequences are to said to “overlap” a given stretch of polynucleotide sequence either in their entirety or along a partial stretch of the poylnucleotide. For example, representative contigs to the polynucleotide sequence 5′-ATGGCTTAGCTT-3′ are 5′-TAGCTTgagtct-3′ and 3′-gtcgacTACCGA-5′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e.g., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, indicates that the segments are arranged so that they function in concert for their intended purposes, e.g., transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of a novel DNA sequence that encodes a novel polypeptide having limited homology to a C. elegans genomic DNA (Wilson, R. et al., Nature 368:32-38, 1994). Analysis of the tissue distribution of the mRNA corresponding to this novel DNA showed that expression was highest in prostate, testis and uterus, followed by medium expression levels in heart, stomach, and liver, and lower in other tissues. The polypeptide has been designated zpep14.

The novel zpep14 polypeptides of the present invention were initially identified by querying an EST database for sequences coding for 2 dibasic sites separated by about 5 to about 30 amino acids. An EST sequence was discovered and predicted to code for part of a secreted protein and the full-length was subsequently isolated.

The full sequence of the zpep14 polypeptide was obtained from a single clone believed to contain it, wherein the clone was obtained from a lymph node tissue library. Other libraries that can also be searched for such sequences include prostate, testis, uterus, heart, liver, stomach, and the like.

The nucleotide sequence of full-length zpep14 is described in SEQ ID NO:1, and its deduced amino acid sequence is described in SEQ ID NO:2. The sequence revealed that zpep14 has a signal sequence, multiple dibasic cleavage sites, and predicted small size (15-40 kD), tissue-specific expression, and lack of long hydrophobic segments, suggesting a small secreted molecule that is in a new class of secreted neuropeptide-like molecules.

Analysis of the DNA encoding zpep14 polypeptide (SEQ ID NO:1) revealed an open reading frame encoding 188 amino acids (SEQ ID NO:2) comprising a predicted signal peptide of 16 amino acid residues (residue 1 (Met) to residue 16 (Ala) of SEQ ID NO:2), and a mature zpep14 polypeptide of 172 amino acids (residue 17 (Arg) to residue 188 (Asn) of SEQ ID NO:2). Moreover, the polypeptide contains multiple dibasic sites that can be the target for post-translational processing of the mature polypeptide, or propeptide, into shorter polypeptide segments that can confer functional and biological properties of zpep14. The dibasic cleavage sites are located at the following residues: Arg₉₃-Arg₉₄, Arg₉₄-Arg₉₅; Arg₁₃₈-Arg₁₃₉; and Lys₁₇₂-Arg₁₇₃. One of skill in the art would recognize that prohormone convertases that can recognize such sites cleave after the C-terminal residue of the paired residues, and then chew back and remove the dibasic residues. Thus, cleavage of the mature zpep14 polypeptide at these dibasic sites reveals several smaller zpep14 polypeptides:

(1) A first polypeptide, referred to hereinafter as “polypeptide-1,” corresponds to amino acid residues 17 (Arg) to amino acid residue 92 (Cys) of SEQ ID NO:2.

(2) A second polypeptide, referred to hereinafter as “polypeptide-2,” corresponds to amino acid residues 96 (Asn) to amino acid residue 137 (Ile) of SEQ ID NO:2. Within polypeptide-2 there are two pairs of cysteine residues, located at residues 110 and 113, and residues 123 and 126 that can form cysteine bonds. The cysteine motif comprises amino acid residues from N-terminal to C-terminal as follows: C-{2}-C-{9}-C-{2}-C, wherein C is a Cys residue, {#} is the number of amino acid residues between Cys residues. In a preferred embodiment, the cysteine motif further comprises a Pro residue after the first Cys residue and a Tyr residue after the fourth Cys residue to give a motif comprising C-P-X-C-{9}-C-{2}-C-Y wherein C is a Cys residue, {#} is the number of amino acid residues between Cys residues, and X is any amino acid.

(3) A third polypeptide, referred to hereinafter as “polypeptide-3,” corresponds to amino acid residues 140 (Gln) to amino acid residue 171 (Gly) of SEQ ID NO:2. Within polypeptide-3 there is an amidation site at Gly₁₇₁.

(4) A fourth polypeptide, referred to hereinafter as “polypeptide-4,” corresponds to amino acid residues 17 (Arg) to amino acid residue 137 (Ile) of SEQ ID NO:2.

(5) A fifth polypeptide, referred to hereinafter as “polypeptide-5,” corresponds to amino acid residues 17 (Arg) to amino acid residue 171 (Gly) of SEQ ID NO:2.

(6) A sixth polypeptide, referred to hereinafter as “polypeptide-6,” corresponds to amino acid residues 96 (Asn) to amino acid residue 171 (Gly) of SEQ ID NO:2.

(7) A C-terminal peptide, referred to hereinafter as “polypeptide-7,” corresponds to amino acid residues 174 (Ile) to amino acid residue 188 (Asn) of SEQ ID NO:2. Polypeptide-7 can be attached to polypeptides-3, -5, or -6 if the dibasic cleavage site at Lys₁₇₂-Arg₁₇₃ is not utilized, which would generate respectfully polypeptide-8 (corresponding to amino acid residues 140 (Gln) to amino acid residue 188 (Asn) of SEQ ID NO:2); the mature zpep14 polypeptide described above; and polypeptide-9 (corresponding to amino acid residues 96 (Asn) to amino acid residue 188 (Asn) of SEQ ID NO:2).

The corresponding polynucleotides encoding the zpep14 polypeptide regions, domains, motifs, residues and sequences described above are as shown in SEQ ID NO:1.

The presence of transmembrane regions, dibasic cleavage sites, cysteine residues, and conserved and low variance motifs generally correlates with or defines important structural regions in proteins. Regions of low variance (e.g., hydrophobic clusters) are generally present in regions of structural importance (Sheppard, P. et al., supra.). Such regions of low variance often contain rare or infrequent amino acids, such as Tryptophan. The regions flanking and between such conserved and low variance motifs may be more variable, but are often functionally significant because they relate to or define important structures and activities such as binding domains, biological and enzymatic activity, signal transduction, cell-cell interaction, tissue localization domains and the like.

The acids in, for example, the cysteine motif of zpep14 can be used as a tool to identify new family members. For instance, reverse transcription-polymerase chain reaction (RT-PCR) can be used to amplify sequences encoding the cysteine motif from above from RNA obtained from a variety of tissue sources or cell lines. In particular, highly degenerate primers designed from the zpep14 sequences are useful for this purpose.

The present invention also provides polynucleotide molecules, including DNA and RNA molecules, that encode the zpep14 polypeptides disclosed herein. Those skilled in the art will readily recognize that, in view of the degeneracy of the genetic code, considerable sequence variation is possible among these polynucleotide molecules. SEQ ID NO:3 is a degenerate DNA sequence that encompasses all DNAs that encode the zpep14 polypeptide of SEQ ID NO:2. Those skilled in the art will recognize that the degenerate sequence of SEQ ID NO:3 also provides all RNA sequences encoding SEQ ID NO:2 by substituting U for T. Thus, zpep14 polypeptide-encoding polynucleotides comprising nucleotide 1 to nucleotide 564 of SEQ ID NO:3 and their RNA equivalents are contemplated by the present invention. Table 1 sets forth the one-letter codes used within SEQ ID NO:3 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | Resolution | Complement | Resolution |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:3, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid Threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO:3 serves as a template for optimizing expression of polynucleotides in various cell types and species commonly used in the art and disclosed herein. Sequences containing preferential codons can be tested and optimized for expression in various species, and tested for functionality as disclosed herein.

Within preferred embodiments of the invention the isolated polynucleotides will hybridize to similar sized regions of SEQ ID NO:1, or a sequence complementary thereto, under stringent conditions. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Numerous equations for calculating Tₘ are known in the art, and are specific for DNA, RNA and DNA-RNA hybrids and polynucleotide probe sequences of varying length (see, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user defined criteria. Such programs can also analyze a given sequence under defined conditions and identify suitable probe sequences. Typically, hybridization of longer polynucleotide sequences, >50 base pairs, is performed at temperatures of about 20-25° C. below the calculated Tₘ. For smaller probes, <50 base pairs, hybridization is typically carried out at the Tₘ or 5-10° C. below. This allows for the maximum rate of hybridization for DNA-DNA and DNA-RNA hybrids. Higher degrees of stringency at lower temperatures can be achieved with the addition of formamide which reduces the Tₘ of the hybrid about 1° C. for each 1% formamide in the buffer solution. Suitable stringent hybridization conditions are equivalent to about a 5 h to overnight incubation at about 42° C. in a solution comprising: about 40-50% formamide, up to about 6×SSC, about 5×Denhardt's solution, zero up to about 10% dextran sulfate, and about 10-20 μg/ml denatured commercially-available carrier DNA. Generally, such stringent conditions include temperatures of 20-70° C. and a hybridization buffer containing up to 6×SSC and 0-50% formamide; hybridization is then followed by washing filters in up to about 2×SSC. For example, a suitable wash stringency is equivalent to 0.1×SSC to 2×SSC, 0.1% SDS, at 55° C. to 65° C. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes. Stringent hybridization and wash conditions depend on the length of the probe, reflected in the Tm, hybridization and wash solutions used, and are routinely determined empirically by one of skill in the art.

As previously noted, the isolated polynucleotides of the present invention include DNA and RNA. Methods for preparing DNA and RNA are well known in the art. In general, RNA is isolated from a tissue or cell that produces large amounts of zpep14 RNA. Such tissues and cells are identified by Northern blotting (Thomas, Proc. Natl. Acad. Sci. USA 77:5201, 1980), and include prostate, uterus, and testis, including whole testis tissue extracts or testicular cells, such as Sertoli cells, Leydig cells, spermatogonia, or epididymis, cells from vas deferens, and cervical cells, although DNA can also be prepared using RNA from other tissues or isolated as genomic DNA. Total RNA can be prepared using guanidinium isothiocyanate extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding zpep14 polypeptides are then identified and isolated by, for example, hybridization or PCR.

A full-length clone encoding zpep14 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to zpep14, receptor fragments, or other specific binding partners.

The polynucleotides of the present invention can also be synthesized using DNA synthesis machines. If chemically synthesized double stranded DNA is required for an application such as the synthesis of a DNA or a DNA fragment, then each complementary strand is made separately, for example via the phosphoramidite method known in the art. The production of short polynucleotides (60 to 80 bp) is technically straightforward and can be accomplished by synthesizing the complementary strands and then annealing them. However, for producing longer polynucleotides (longer than about 300 bp), special strategies are usually employed. For example, synthetic DNAs (double-stranded) are assembled in modular form from single-stranded fragments that are from 20 to 100 nucleotides in length. One method for building a synthetic DNA involves producing a set of overlapping, complementary oligonucleotides. Each internal section of the DNA has complementary 3′ and 5′ terminal extensions designed to base pair precisely with an adjacent section. After the DNA is assembled, the process is completed by ligating the nicks along the backbones of the two strands. In addition to the protein coding sequence, synthetic DNAs can be designed with terminal sequences that facilitate insertion into a restriction endonuclease site of a cloning vector. Alternative ways to prepare a full-length DNA are also known in the art. See Glick and Pasternak, Molecular Biotechnology, Principles & Applications of Recombinant DNA, (ASM Press, Washington, D.C. 1994); Itakura et al., Annu. Rev. Biochem. 53: 323-56, 1984 and Climie et al., Proc. Natl. Acad. Sci. USA 87:633-7,1990.

The present invention further provides counterpart polypeptides and polynucleotides from other species (orthologs). These species include, but are not limited to mammalian, avian, amphibian, reptile, fish, insect and other vertebrate and invertebrate species. Of particular interest are zpep14 polypeptides from other mammalian species, including murine, porcine, ovine, bovine, canine, feline, equine, and other primate polypeptides. Orthologs of human zpep14 can be cloned using information and compositions provided by the present invention in combination with conventional cloning techniques. For example, a cDNA can be cloned using mRNA obtained from a tissue or cell type that expresses zpep14 as disclosed herein. Suitable sources of mRNA can be identified by probing Northern blots with probes designed from the sequences disclosed herein. A library is then prepared from mRNA of a positive tissue or cell line. A zpep14-encoding cDNA can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the representative human zpep14 sequence disclosed herein. Within an additional method, the cDNA library can be used to transform or transfect host cells, and expression of the cDNA of interest can be detected with an antibody to zpep14 polypeptide. Similar techniques can also be applied to the isolation of genomic clones.

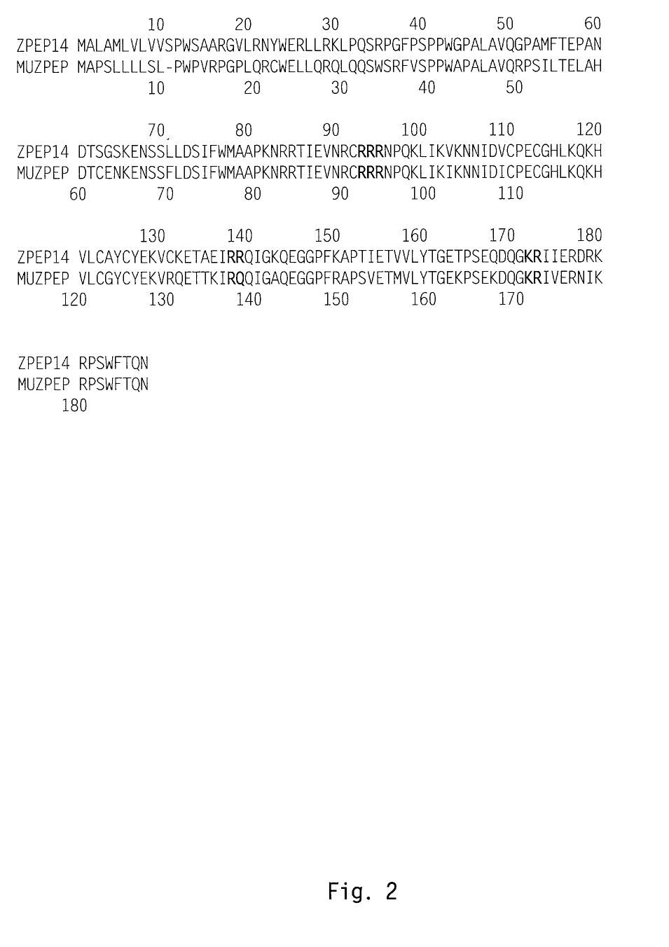

The mouse ortholog of zpep14, mouse zpep14, was isolated and the polynucleotide sequence is shown in SEQ ID NO:17. The mouse zpepe14 polypeptide sequence is shown in SEQ ID NO:18. Multiple alignment of the human and mouse zpep14 polypeptides reveals that the mouse sequence includes all the corresponding dibasic cleavage sites except the Arg₁₃₈-Arg₁₃₉ site (See, FIG. 2). Analysis of the DNA encoding mouse zpep14 polypeptide (SEQ ID NO:17) revealed an open reading frame encoding 187 amino acids (SEQ ID NO:18) comprising a predicted signal peptide of 16 amino acid residues (residue 1 (Met) to residue 16 (Pro) of SEQ ID NO:18), and a mature mouse zpep14 polypeptide of 171 amino acids (residue 17 (Gly) to residue 187 (Asn) of SEQ ID NO:18). Moreover, the polypeptide contains multiple dibasic sites that can be the target for post-translational processing of the mature polypeptide, or pro-peptide, into shorter polypeptide segments that can confer functional and biological properties of mouse zpep14. The dibasic cleavage sites are located at the following residues: Arg₉₂-Arg₉₃, Arg₉₃-Arg₉₄; and Lys₁₇₁-Arg₁₇₂. One of skill in the art would recognize that prohormone convertases that can recognize such sites cleave after the C-terminal residue of the paired residues, and then chew back and remove the dibasic residues. Thus, cleavage of the mature mouse zpep14 polypeptide at these dibasic sites reveals several smaller mouse zpep14 polypeptides:

(1) A first polypeptide, referred to hereinafter as “polypeptide-1m,” corresponds to amino acid residues 17 (Gly) to amino acid residue 91 (Cys) of SEQ ID NO:18.

(2) A second polypeptide, referred to hereinafter as “polypeptide-5m,” corresponds to amino acid residues 17 (Gly) to amino acid residue 170 (Gly) of SEQ ID NO:2. Within polypeptide-5m there are two pairs of cysteine residues, located at residues 109 and 112, and residues 122 and 125 that can form cysteine bonds. The cysteine motif comprises amino acid residues from N-terminal to C-terminal as follows: C-{2}-C-{9}-C-{2}-C, wherein C is a Cys residue, {#} is the number of amino acid residues between Cys residues. In a preferred embodiment, the cysteine motif further comprises a Pro residue after the first Cys residue and a Tyr residue after the fourth Cys residue to give a motif comprising C-P-X-C-{9}-C-{2}-C-Y wherein C is a Cys residue, {#} is the number of amino acid residues between Cys residues, and X is any amino acid. Within polypeptide-5m there is an amidation site at Gly₁₇₀.

(6) A third polypeptide, referred to hereinafter as “polypeptide-6m,” corresponds to amino acid residues 95 (Asn) to amino acid residue 170 (Gly) of SEQ ID NO:18. Polypeptide-6m also contains the cysteine motif and amidation site described above.

(7) A C-terminal peptide, referred to hereinafter as “polypeptide-7m,” corresponds to amino acid residues 173 (Ile) to amino acid residue 187 (Asn) of SEQ ID NO:18. Polypeptide-7m can be attached to polypeptides-5m, or -6m if the dibasic cleavage site at Lys₁₇₁-Arg₁₇₂ is not utilized, which would generate respectfully the mature mouse zpep14 polypeptide described above; and polypeptide-9m (corresponding to amino acid residues 95 (Asn) to amino acid residue 187 (Asn) of SEQ ID NO:18).

Those skilled in the art will recognize that the sequence disclosed in SEQ ID NO:1 represents a single allele of human zpep14 and that allelic variation and alternative splicing are expected to occur. Allelic variants of this sequence can be cloned by probing cDNA or genomic libraries from different individuals according to standard procedures. Allelic variants of the DNA sequence shown in SEQ ID NO:1, including those containing silent mutations and those in which mutations result in amino acid sequence changes, are within the scope of the present invention, as are proteins which are allelic variants of SEQ ID NO:2. cDNAs generated from alternatively spliced mRNAs, which retain the properties of the zpep14 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

The corresponding polynucleotides encoding the mouse zpep14 polypeptide regions, domains, motifs, residues and sequences described above are as shown in SEQ ID NO:17.

The present invention also provides isolated zpep14 polypeptides that are substantially similar to the polypeptides of SEQ ID NO:2 and their orthologs. The term “substantially similar” is used herein to denote polypeptides having 70%, preferably 75%, more preferably at least 80%, sequence identity to the sequences shown in SEQ ID NO:2 or their orthologs. Such polypeptides will more preferably be at least 90% identical, and most preferably 95% or more identical to SEQ ID NO:2 or its orthologs.) Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-9, 1992. Briefly, two amino acid sequences are aligned to optimize the alignment scores using a gap opening penalty of 10, a gap extension penalty of 1, and the “blosum 62” scoring matrix of Henikoff and Henikoff (supra.) as shown in Table 3 (amino acids are indicated by the standard one-letter codes). The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant zpep14. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

The BLOSUM62 table (Table 3) is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed below), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g. 2 or 3).

Variant zpep14 polypeptides or substantially homologous zpep14 polypeptides are characterized as having one or more amino acid substitutions, deletions or additions. These changes are preferably of a minor nature, that is conservative amino acid substitutions (see Table 4) and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. The present invention thus includes polypeptides of from about 14 to about 200 amino acid residues that comprise a sequence that is at least 80%, preferably at least 90%, and more preferably 95% or more identical to the corresponding region of SEQ ID NO:2. Polypeptides comprising affinity tags can further comprise a proteolytic cleavage site between the zpep14 polypeptide and the affinity tag. Preferred such sites include thrombin cleavage sites and factor Xa cleavage sites.

TABLE 4
Conservative amino acid substitutions

| | |
|---|---|
| Basic | arginine |
| | lysine |
| | histidine |
| Acidic | glutamic acid |
| | aspartic acid |
| Polar | glutamine |
| | asparagine |
| Hydrophobic | leucine |
| | isoleucine |
| | valine |
| Aromatic | phenylalanine |
| | tryptophan |
| | tyrosine |
| Small | glycine |
| | alanine |
| | serine |
| | threonine |
| | methionine |

The present invention further provides a variety of other polypeptide fusions and related multimeric proteins comprising one or more polypeptide fusions. For example, a zpep14 polypeptide can be prepared as a fusion to a dimerizing protein as disclosed in U.S. Pat. Nos. 5,155,027 and 5,567,584. Preferred dimerizing proteins in this regard include immunoglobulin constant region domains. Immunoglobulin-zpep14 polypeptide fusions can be expressed in genetically engineered cells to produce a variety of multimeric zpep14 analogs. Auxiliary domains can be fused to zpep14 polypeptides to target them to specific cells, tissues, or macromolecules (e.g., collagen). For example, a zpep14 polypeptide or protein can be targeted to a predetermined cell type by fusing a zpep14 polypeptide to a ligand that specifically binds to a receptor on the surface of the target cell. In this way, polypeptides and proteins can be targeted for therapeutic or diagnostic purposes. A zpep14 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for zpep14 amino acid residues.

Essential amino acids in the polypeptides of the present invention can be identified according to procedures known in the art, such as site-directed mutagenesis or alanine-scanning mutagenesis (Cunningham and Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699-708, 1996. Sites of ligand-receptor or other biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related polypeptide sequences or proteins.

Determination of amino acid residues that are within regions or domains that are critical to maintaining structural integrity can be determined. Within these regions one can determine specific residues that will be more or less tolerant of change and maintain the overall tertiary structure of the molecule. Methods for analyzing sequence structure include, but are not limited to, alignment of multiple sequences with high amino acid or nucleotide identity and computer analysis using available software (e.g., the Insight II® viewer and homology modeling tools; MSI, San Diego, Calif.), secondary structure propensities, binary patterns, complementary packing and buried polar interactions (Barton, Current Opin. Struct. Biol. 5:372-376, 1995 and Cordes et al., Current Opin. Struct. Biol. 6:3-10, 1996). In general, when designing modifications to molecules or identifying specific fragments determination of structure will be accompanied by evaluating activity of modified molecules.

Amino acid sequence changes are made in zpep14 polypeptides so as to minimize disruption of higher order structure essential to biological activity. For example, when the zpep14 polypeptide comprises one or more conserved structures, changes in amino acid residues will be made so as not to disrupt the structures and other components of the molecule where changes in conformation abate some critical function, for example, binding of the molecule to its binding partners. The effects of amino acid sequence changes can be predicted by, for example, computer modeling as disclosed herein or determined by analysis of crystal structure (see, e.g., Lapthorn et al., Nat. Struct. Biol. 2:266-268, 1995). Other techniques that are well known in the art compare folding of a variant protein to a standard molecule (e.g., the native protein). For example, comparison of the cysteine pattern in a variant and standard molecules can be made. Mass spectrometry and chemical modification using reduction and alkylation provide methods for determining cysteine residues which are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). It is generally believed that if a modified molecule does not have the same disulfide bonding pattern as the standard molecule folding would be affected. Another well known and accepted method for measuring folding is circular dichrosism (CD). Measuring and comparing the CD spectra generated by a modified molecule and standard molecule is routine (Johnson, Proteins 7:205-214, 1990). Crystallography is another well known method for analyzing folding and structure. Nuclear magnetic resonance (NMR), digestive peptide mapping and epitope mapping are also known methods for analyzing folding and structural similarities between proteins and polypeptides (Schaanan et al., Science 257:961-964, 1992).

A Hopp/Woods hydrophilicity profile of the zpep14 protein sequence as shown in SEQ ID NO:2 can be generated (Hopp et al., Proc. Natl. Acad. Sci.78:3824-3828, 1981; Hopp, J. Immun. Meth. 88:1-18, 1986 and Triquier et al., Protein Engineering 11:153-169, 1998). The profile is based on a sliding six-residue window. Buried G, S, and T residues and exposed H, Y, and W residues were ignored. For example, in zpep14, hydrophilic regions include: (1) amino acid number 90 (Asn) to amino acid number 95 (Arg) of SEQ ID NO:2; (2) amino acid number 128 (Glu) to amino acid number 133 (Glu) of SEQ ID NO:2; (3) amino acid number 167 (Glu) to amino acid number 1 72 (Lys) of SEQ ID NO:2; (4) amino acid number 175 (Ile) to amino acid number 180 (Lys) of SEQ ID NO:2; and (5) amino acid number 176 (Glu) to amino acid number 181 (Arg) of SEQ ID NO:2.

Those skilled in the art will recognize that hydrophilicity or hydrophobicity will be taken into account when designing modifications in the amino acid sequence of a zpep14 polypeptide, so as not to disrupt the overall structural and biological profile. Of particular interest for replacement are hydrophobic residues selected from the group consisting of Val, Leu and Ile or the group consisting of Met, Gly, Ser, Ala, Tyr and Trp. For example, residues tolerant of substitution could include these hydrophobic residues as shown in SEQ ID NO:2. Cysteine residues in the cysteine motifs of SEQ ID NO:2 and SEQ ID NO:18, described herein, will be relatively intolerant of substitution.

The identities of essential amino acids can also be inferred from analysis of sequence similarity between family members with zpep14. Using methods such as “FASTA” analysis described previously, regions of high similarity are identified within a family of proteins and used to analyze amino acid sequence for conserved regions. An alternative approach to identifying a variant zpep14 polynucleotide on the basis of structure is to determine whether a nucleic acid molecule encoding a potential variant zpep14 polynucleotide can hybridize to a nucleic acid molecule having the nucleotide sequence of SEQ ID NO:1, as discussed above.

Other methods of identifying essential amino acids in the polypeptides of the present invention are procedures known in the art, such as site-directed mutagenesis or alanine-scanning mutagenesis (Cunningham and Wells, Science 244:1081 (1989), Bass et al., Proc. Natl Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity as disclosed below to identify amino acid residues that are critical to the activity of the molecule. See also, Hilton et al, J. Biol. Chem. 271:4699 (1996).

The present invention also includes functional fragments of zpep14 polypeptides and nucleic acid molecules encoding such functional fragments. A “functional” zpep14 or fragment thereof defined herein is characterized by its proliferative or differentiating activity, by its ability to induce or inhibit specialized cell functions, or by its ability to bind specifically to an anti-zpep14 antibody or zpep14 receptor (either soluble or immobilized). As previously described herein, zpep14 is characterized by several cleavage sites that generate a number of bioactive zpep14 peptides. Thus, the present invention further provides fusion proteins encompassing: (a) polypeptide molecules comprising one or more of the of the zpep14 peptides described above; and (b) functional fragments comprising one or more of these peptides. The other polypeptide portion of the fusion protein may be contributed by another peptide hormone, such as insulin, glucagon, POMC, growth hormone, neuropeptide hormones, and the like, or by a non-native and/or an unrelated secretory signal peptide that facilitates secretion of the fusion protein.

Routine deletion analyses of nucleic acid molecules can be performed to obtain functional fragments of a nucleic acid molecule that encodes a zpep14 polypeptide. As an illustration, DNA molecules having the nucleotide sequence of SEQ ID NO:1 or fragments thereof, can be digested with Bal31 nuclease to obtain a series of nested deletions. These DNA fragments are then inserted into expression vectors in proper reading frame, and the expressed polypeptides are isolated and tested for zpep14 activity, or for the ability to bind anti-zpep14 antibodies or zpep14 receptor. One alternative to exonuclease digestion is to use oligonucleotide-directed mutagenesis to introduce deletions or stop codons to specify production of a desired zpep14 fragment. Alternatively, particular fragments of a zpep14 polynucleotide can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993); Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987); Herschman, “The EGF Receptor,” in Control of Animal Cell Proliferation 1, Boynton et al., (eds.) pages 169-199 (Academic Press 1985); Coumailleau et al., J. Biol. Chem. 270:29270 (1995); Fukunaga et al., J. Biol. Chem. 270:25291 (1995); Yamaguchi et al., Biochem. Pharmacol. 50:1295 (1995); and Meisel et al., Plant Molec. Biol. 30:1 (1996).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed zpep14 DNA and polypeptide sequences can be generated through DNA shuffling as disclosed by Stemmer, Nature 370:389-91, 1994, Stemmer, Proc. Natl. Acad. Sci. USA 91:10747-51, 1994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode active polypeptides (e.g., secreted and detected by antibodies, binding assays, or measured by a signal transduction type assay) can be recovered from the host cells and rapidly sequenced using modern equipment. These methods allow the rapid determination of the importance of individual amino acid residues in a polypeptide of interest, and can be applied to polypeptides of unknown structure.

Using the methods discussed herein, one of ordinary skill in the art can identify and/or prepare a variety of polypeptides that are substantially similar to SEQ ID NO:2 or allelic variants thereof and retain the properties of the wild-type protein. For example, using the methods described above, one could identify a receptor binding domain on zpep14; an extracellular ligand-binding domain of a receptor for zpep14; heterodimeric and homodimeric binding domains; other functional or structural domains; affinity tags; or other domains important for protein-protein interactions or signal transduction. Such polypeptides may also include additional polypeptide segments as generally disclosed above.

For any zpep14 polypeptide, including variants and fusion proteins, one of ordinary skill in the art can readily generate a fully degenerate polynucleotide sequence encoding that variant using the information set forth in Tables 1 and 2 above.

The zpep14 polypeptides of the present invention, including full-length polypeptides, polypeptides-1 thorough 9 described herein, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a zpep14 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a zpep14 polypeptide into the secretory pathway of a host cell, a secretory signal sequence (also known as a leader sequence, prepro sequence or pre sequence) is provided in the expression vector. The secretory signal sequence may be that of zpep14, or may be derived from another secreted protein (e.g., t-PA) or synthesized de novo. The secretory signal sequence is operably linked to the zpep14 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

Alternatively, the secretory signal sequence contained in the polypeptides of the present invention is used to direct other polypeptides into the secretory pathway. The present invention provides for such fusion polypeptides. A signal fusion polypeptide can be made wherein a secretory signal sequence derived from residue 1 (Met) to residue 16 (Ala) of SEQ ID NO:2 is operably linked to a DNA sequence encoding another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein. Such fusions may be used in vivo or in vitro to direct peptides through the secretory pathway.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Manassas, Va. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. The second method of making recombinant baculovirus utilizes a transposon-based system described by Luckow (Luckow, V. A, et al., J Virol 67:4566-79, 1993). This system is sold in the Bac-to-Bac™ kit (Life Technologies, Rockville, Md.). This system utilizes a transfer vector, pFastBac1™ (Life Technologies) containing a Tn7 transposon to move the DNA encoding the zpep14 polypeptide into a baculovirus genome maintained in E. coli as a large plasmid called a “bacmid.” The pFastBac1™ transfer vector utilizes the AcNPV polyhedrin promoter to drive the expression of the gene of interest, in this case zpep14. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol. Chem. 270:1543-9, 1995. In such transfer vector constructs, a short or long version of the basic protein promoter can be used. Moreover, transfer vectors can be constructed which replace the native zpep14 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native zpep14 secretory signal sequence. In addition, transfer vectors can include an in-frame fusion with DNA encoding an epitope tag at the C- or N-terminus of the expressed zpep14 polypeptide, for example, a Glu-Glu epitope tag (Grussenmeyer, T. et al., Proc. Natl. Acad. Sci. 82:7952-4, 1985). Using a technique known in the art, a transfer vector containing zpep14 is transformed into E. Coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses zpep14 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D. R. et al., ibid.; Richardson, C. D., ibid.). Subsequent purification of the zpep14 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a zpep14 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

It is preferred to purify the polypeptides of the present invention to ≧80% purity, more preferably to ≧90% purity, even more preferably ≧95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant zpep14 polypeptides (or chimeric zpep14 polypeptides) can be purified using fractionation and/or conventional purification methods and media. Ammonium sulfate precipitation and acid or chaotrope extraction may be used for fractionation of samples. Exemplary purification steps can include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their structural and biological properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39). Within additional embodiments of the invention, a fusion of the polypeptide of interest and an affinity tag (e.g., maltose-binding protein, an immunoglobulin domain) may be constructed to facilitate purification.

Moreover, using methods described in the art, polypeptide fusions, or hybrid zpep14 proteins, are constructed using regions or domains of zpep14 in combination with those of paralogs, orthologs, or heterologous proteins (Sambrook et al., ibid., Altschul et al., ibid., Picard. D., Cur. Opin. Biology, 5:511-515, 1994, and references therein). These methods allow the determination of the biological importance of larger domains or regions in a polypeptide of interest. Such hybrids may alter reaction kinetics, binding, constrict or expand the substrate specificity, or alter tissue and cellular localization of a polypeptide, and can be applied to polypeptides of unknown structure.

Fusion polypeptides can be prepared by methods known to those skilled in the art by preparing each component of the fusion protein and chemically conjugating them. Alternatively, a polynucleotide encoding one or more components of the fusion protein in the proper reading frame can be generated using known techniques and expressed by the methods described herein. For example, part or all of a domain(s) conferring a biological function may be swapped between zpep14 of the present invention with the functionally equivalent domain(s) from another family member. Such domains include, but are not limited to the secretory signal sequence, and polypeptides-1 through 9 described herein. Such fusion proteins would be expected to have a biological functional profile that is the same or similar to polypeptides of the present invention or other known family proteins or to a heterologous protein, depending on the fusion constructed. Moreover, such fusion proteins may exhibit other properties as disclosed herein.

Standard molecular biological and cloning techniques can be used to swap the equivalent domains between the zpep14 polypeptide and those polypeptides to which they are fused. Generally, a DNA segment that encodes a domain of interest, e.g., a zpep14 polypeptide-1 through -9, or motif described herein, is operably linked in frame to at least one other DNA segment encoding an additional polypeptide and inserted into an appropriate expression vector, as described herein. Generally DNA constructs are made such that the several DNA segments that encode the corresponding regions of a polypeptide are operably linked in frame to make a single construct that encodes the entire fusion protein, or a functional portion thereof. For example, a DNA construct would encode from N-terminus to C-terminus a fusion protein comprising a signal polypeptide followed by a mature polypeptide; or a DNA construct would encode from N-terminus to C-terminus a fusion protein comprising a signal polypeptide followed by polypeptide-1, followed by polypeptide-2, followed by polypeptide-3, or as interchanged with equivalent regions from another protein. Such fusion proteins can be expressed, isolated, and assayed for activity as described herein.

Protein refolding (and optionally reoxidation) procedures may be advantageously used. Zzpep14 polypeptides or fragments thereof may also be prepared through chemical synthesis. zpep14 polypeptides may be monomers or multimers; glycosylated or non-glycosylated; pegylated or non-pegylated; and may or may not include an initial methionine amino acid residue.

Polypeptides of the present invention can also be synthesized by exclusive solid phase synthesis, partial solid phase methods, fragment condensation or classical solution synthesis. Methods for synthesizing polypeptides are well known in the art. See, for example, Merrifield, J. Am. Chem. Soc. 85:2149, 1963; Kaiser et al., Anal. Biochem. 34:595, 1970. After the entire synthesis of the desired peptide on a solid support, the peptide-resin is washed with a reagent which cleaves the polypeptide from the resin and removes most of the side-chain protecting groups. Such methods are well established in the art.

The activity of molecules of the present invention can be measured using a variety of assays that measure cell differentiation and proliferation as well as assays that measure cell contractility and cardiovascular function. Such assays are well known in the art.

Several tissues in which zpep14 is highly and moderately expressed are tissues that contract. For example contractile tissues in which zpep14 is expressed include uterus; tissues in testis, e.g., vas deferens; prostate tissues; gastrointestinal tissues, e.g., colon and small intestine; and heart. The effects of zpep14 polypeptide, its antagonists and agonists, on tissue contractility can be measured in vitro using a tensiometer with or without electrical field stimulation. Such assays are known in the art and can be applied to tissue samples, such as aortic rings, vas deferens, ilium, uterine and other contractile tissue samples, as well as to organ systems, such as atria, and can be used to determine whether zpep14 polypeptide, its agonists or antagonists, enhance or depress contractility. Molecules of the present invention are hence useful for treating dysfunction associated with contractile tissues or can be used to suppress or enhance contractility in vivo. As such, molecules of the present invention have utility in treating cardiovascular disease, infertility, in vitro fertilization, birth control, treating impotence or other male reproductive dysfunction, as well as inducing birth.

The effect of the zpep14 polypeptides, antagonists and agonists of the present invention on contractility of tissues including uterus, prostate, testis, gastrointestinal tissues, and heart can be measured in a tensiometer that measures contractility and relaxation in tissues. See, Dainty et al., J. Pharmacol. 100:767, 1990; Rhee et al., Neurotox. 16: 179, 1995; Anderson, M. B., Endocrinol. 114:364-368, 1984; and Downing, S. J. and Sherwood, O. D, Endocrinol. 116:1206-1214, 1985. For example, measuring vasodilatation of aortic rings is well known in the art. Briefly, aortic rings are taken from 4 month old Sprague Dawley rats and placed in a buffer solution, such as modified Krebs solution (118.5 mM NaCl, 4.6 mM KCl, 1.2 mM MgSO₄.7H₂O, 1.2 mM KH₂PO₄, 2.5 mM CaCl₂.2H₂O, 24.8 mM NaHCO₃ and 10 mM glucose). One of skill in the art would recognize that this method can be used with other animals, such as rabbits, other rat strains, Guinea pigs, and the like. The rings are then attached to an isometric force transducer (Radnoti Inc., Monrovia, Calif.) and the data recorded with a Ponemah physiology platform (Gould Instrument systems, Inc., Valley View, Ohio) and placed in an oxygenated (95% O₂, 5% CO₂) tissue bath containing the buffer solution. The tissues are adjusted to 1 gram resting tension and allowed to stabilize for about one hour before testing. The integrity of the rings can be tested with norepinepherin (Sigma Co., St. Louis, Mo.) and Carbachol, a muscarinic acetylcholine agonist (Sigma Co.). After integrity is checked, the rings are washed three times with fresh buffer and allowed to rest for about one hour. To test a sample for vasodilatation, or relaxation of the aortic ring tissue, the rings are contracted to two grams tension and allowed to stabilize for fifteen minutes. A zpep14 polypeptide sample is then added to 1, 2 or 3 of the 4 baths, without flushing, and tension on the rings recorded and compared to the control rings containing buffer only. Enhancement or relaxation of contractility by zpep14 polypeptides, their agonists and antagonists is directly measured by this method, and it can be applied to other contractile tissues such as uterus, prostate, and testis.

The activity of molecules of the present invention can be measured using a variety of assays that measure stimulation of gastrointestinal cell contractility, modulation of nutrient uptake and/or secretion of digestive enzymes. Of particular interest are changes in contractility of smooth muscle cells. For example, the contractile response of segments of mammalian duodenum or other gastrointestinal smooth muscles tissue (Depoortere et al., J. Gastrointestinal Motility 1:150-159, 1989, incorporated herein by reference). An exemplary in vivo assay uses an ultrasonic micrometer to measure the dimensional changes radially between commissures and longiturdinally to the plane of the valve base (Hansen et al., Society of Thoracic Surgeons 60:S384-390, 1995).

Gastric motility is generally measured in the clinical setting as the time required for gastric emptying and subsequent transit time through the gastrointestinal tract. Gastric emptying scans are well known to those skilled in the art, and briefly, comprise use of an oral contrast agent, such as barium, or a radiolabeled meal. Solids and liquids can be measured independently. A test food or liquid is radiolabeled with an isotope (e.g. ^{99m}Tc), and after ingestion or administration, transit time through the gastrointestinal tract and gastric emptying are measured by visualization using gamma cameras (Meyer et al., Am. J. Dig. Dis. 21:296, 1976; Collins et al., Gut 24:1117, 1983; Maughan et al., Diabet. Med. 13 9 Supp. 5:S6-10, 1996 and Horowitz et al., Arch. Intern. Med. 145:1467-1472, 1985). These studies may be performed before and after the administration of a promotility agent to quantify the efficacy of the drug.

As a polypeptide or peptide expressed in heart, zpep14 could be useful as modulator of blood pressure, muscle tension or and osmotic balance. For example, blood pressure modification is important in situations such as heart attack, stroke, traumatic shock, surgery, and any number of bleeding complications. As a modulator of blood pressure, muscle tension or and osmotic balance, zpep14 may modulate contractility in the organ systems and tissues that it effects. Thus, The activity of molecules of the present invention can be measured using a variety of assays that measure cell contractility and discussed below. Such assays are well known in the art, and described herein.

Many peptide hormones, such as those within family of gut-brain peptides, are associated with neurological and CNS functions as well as cardiovascular functions. For example, NPY, a peptide with receptors in both the brain and the gut has been shown to stimulate appetite when administered to the central nervous system (Gehlert, Life Sciences 55(6):551-562, 1994). Moreover, NPY has been implicated in cardiovascular effects such as increased sympathetic nerve activity in heart, which is associated with heart failure, as well as hypotension, and changes in blood pressure and vagal action (Feng, Q. et al Acta. Physiol. Scand. 166:285-291, 1999; McLean, K J. Et al. Neuroscience 92:1377-1387, 1999; Potter, E K et al; Regul. Pept. 25:167-177, 1989; Gardiner, S M Brain Res. Brain Res. Review 14:79-116, 1989). Moreover, other peptide hormones such as motilin, have immunoreactivity identified in different regions of the brain, particularly the cerebellum, and in the pituitary (Gasparini et al., Hum. Genetics 94(6):671-674, 1994). Motilin has been found to coexist with neurotransmitter γ-aminobutyric acid in cerebellum (Chan-Patay, Proc. Sym. 50th Anniv. Meet. Br. Pharmalog. Soc.:1-24, 1982). Physiological studies have provided some evidence that motilin has an affect on feeding behavior (Rosenfield et al., Phys. Behav. 39(6):735-736, 1987), bladder control, pituitary growth hormone release.

Examples such as NPY and motilin emphasize the importance and broad activity of peptide hormones in the human body, and their impact on normal physiological function and disease. Peptide hormones are involved in regulatory aspects of cardiovascular regulation and homeostasis, digestion, brain, neuronal and other organ functions. Various peptide hormones have been shown to be involved in control of blood pressure, heart rate, arrhythmia, osmotic balance, influencing the release and action of cardiovascular transmitters, vasoconstriction and vasodilatation, vasoconstriction resulting in myocardial ischemia, vasomotor tone, contractility, food intake, respiration, behavior, and pain modulation, and the like. As a peptide hormone, zpep14 and polypeptides 1-9 may similarly exert effects in heart, or other tissues in which it is expressed, or freely circulate through the body and exert effects elsewhere. Thus, zpep14 polypeptide or zpep14 peptides can regulate positively or negatively various physiological functions, or cause the release of other regulatory hormones from the heart, gut, CNS and other organs or tissues. Assays and models to test for such zpep14 activity are well known in the art and described herein. For example, see amongst other methods known in the art: Feng, Q. et al supra. (pithed rat heart failure model to assess vascular sympathetic nerve activity); Horackova, et al., Cell Tissue Res. 297:409-421, 1999 (guinea pig atria model); McLean, K J. Et al. supra. (CNS response to hypotensive challenge to assess neuron response or activation within cardiovascular control); Potter, E K et al; supra. (Testing effects of polypeptides and peptide fragments on blood pressure and vagal action at the heart); Maturi, M F et al., J. Clin. Invest 83:1217-1224 (myocardial ischemia and coronary constriction model in dogs); Haass, M. et al., Naunyn Schmiedebergs Arch. Pharmacol. 25 339:71-78, 1989 (pre-synaptic modulation in in situ perfused guinea pig heart); Hassall, C J, nad Burnstock, G. Neurosci. Lett. 52:111-115, 1984 (Cultured Guinea pig atria to study intrinsic innnervation); Lundberg, J M. Et al., Acta. Physiol. Scand. 121:325-332, 1984 (effect of peptide on muscle tone, and autonomic transmission in Guinea pig atrium, vas deferens, urinary bladder, portal vein, and trachea); Mathias, C J J. Neurosci. Methods 34:193-200, 1990 (effect of food in take on cardiovascular control); Miyata, A. et al., Ann. N.Y. Acad. Sci. 865:73-81, 1998 (effect of peptides on rat aortic smooth muscle cell proliferation); Saita, M. et al., Am. J. Physiol. 274:R979-984, 1998 (Effects of centrally administered peptide on blood pressure, heart rate, renal sympathetic nerve activity in rats); Krowicki, Z K et al., Am. J. Physiol. 272:G1221-1229, 1997 (vagally mediated gastric motor excitation); Hall. M E et al., Brain Res. 497:280-290, 1989 (microinjection of peptides into the nucleus of the solitary tract (NTS) and effects on cardiovascular function).

Moreover, immunohistochemical and immunolabeling methods known in the art and described herein can be used to assess zpep14 polypeptide and peptide influence on the release and of cardiovascular effectors and other cardiovascular function, as well as interactions between zpep14 polypeptides and peptides with other peptide effectors, such as VIP, NPY and other peptides (Wharton, J, and Gulbenkian S. Experientia Suppl. 56:292-316, 1989; and Forsgren, S. Cell Tissue Res. 256:125-135, 1989). As such, labeled inventive zpep14 polypeptides, peptides, and antibodies can be used to assess these interactions. In addition, such labeled zpep14 polypeptides, peptides, and antibodies can be used as diagnostics to assess human disease in comparison to normal controls, and described herein. Such histologic, immunohistochemical and immunolabeling methods and the like can be used in conjunction with the in vivo models described above and herein.

The cardiac activity of molecules of the present invention may be measured using a Langendorff assay. This preferred assay measures ex vivo cardiac function for an experimental animal, and is well known in the art. Experimental animals are, for example but not limited to, rats, rabbits and guinea pigs. Chronic effects on heart tissue can be measured after treating a test animal with zpep14 polypeptide for 1 to 7 days, or longer. Control animals will have only received buffer. After treatment, the heart is removed and perfused retrograde through the aorta. During perfusion, several physiologic parameters are measured: coronary blood flow per time, left ventricular (LV) pressures, and heart rate. These perameters directly reflect cardiac function. Changes in these parameters, as measured by the Langendorff assay, following in vivo treatment with zpep14 polypeptide relative to control animals indicates a chronic effect of the polypeptide on heart function. Moreover, the Langendorff assay can also be employed to measure the acute effects of zpep14 polypeptide on heart. In such application, hearts from untreated animals are used and zpep14 polypeptide is added to the perfusate in the assay. The parameters assessed above are measured and compared with the results from control hearts where zpep14 polypeptide was omitted from the perfusate. Differences in heart rate, change in pressure per time, and/or coronary blood flow indicate an acute effect of the molecules of the present invention on heart function.

The activity of molecules of the present invention may also be measured using a variety of assays that measure ion channel activity. Of particular interest is measuring ion transfer cross cell membranes. Such assays are well known in the art. Specific assays to assess the activity of novel ion channels or their regulators include, but are not limited to, bioassays measuring voltage-dependent conductance in Xenopus laevis oocytes (see, Rudy, B., Iverson, L. E., eds., Meth. Enzymol., vol. 207, Academic Press, San Diego, Calif., 1992; Hamill, O. P et al., Pfluegers Arch. 391:85-100, 1981; Moorman, J. R. et al., J. Biol. Chem. 267:14551-14554, 1992; Durieux, M. E., et al., Am. J. Physiol. 263:C896-C900, 1992). This method involves injecting in vitro expressed mRNAs into isolated oocytes and assessing voltage-dependent conductance using a patch-clamp technique. An ion channel or its regulator may increase voltage-dependent conductance in this assay system. This system may be applied to other cell types, such as insect and mammalian cells (see, Rudy, B., Iverson, L. E., eds., ibid.). Other assays involve measuring ion channel activity indirectly in mammalian or other cell types, through the use of a chelator dye, such as Fura2 (See, for example, James-Kracke M. R., J. Gen. Physiol. 99:41-62, 1992; Raghu, P. et al., Gene 190:151-156, 1997). Ion channel activity can also be monitored by using a radiolabeled ion, such as a ¹²⁵I efflux assay (Xia, Y. et al., J. Membr. Biol. 151:269-278, 1996). Other assays involve measuring changes in gene expression in mammalian cells signaled by ion flux or ion channel phosphorylation; for example, by driving expression of a measurable reporter gene, e.g. luciferase, under a suitable promoter as disclosed herein.

The molecules of the present invention may be useful for proliferation of cardiac tissue cells, such as cardiac myocytes or myoblasts; skeletal myocytes or myoblasts and smooth muscle cells; chrondrocytes; endothelial cells; adipocytes and osteoblasts in vitro. For example, molecules of the present invention are useful as components of defined cell culture media, and may be used alone or in combination with other cytokines and hormones to replace serum that is commonly used in cell culture. Molecules of the present invention are particularly useful in specifically promoting the growth and/or development of myocytes in culture, and may also prove useful in the study of cardiac myocyte hyperplasia and regeneration.

The polypeptides, nucleic acids and/or antibodies of the present invention may be used in treatment of disorders associated with myocardial infarction, congestive heart failure, hypertrophic cardiomyopathy and dilated cardiomyopathy. Molecules of the present invention may also be useful for limiting infarct size following a heart attack, aiding in recovery after heart transplantation, promoting angiogenesis and wound healing following angioplasty or endarterectomy, to develop coronary collateral circulation, for revascularization in the eye, for complications related to poor circulation such as diabetic foot ulcers, for stroke, following coronary reperfusion using pharmacologic methods, and other indications where angiogenesis is of benefit. Molecules of the present invention may be useful for improving cardiac function, either by inducing cardiac myocyte neogenesis and/or hyperplasia, by inducing coronary collateral development, or by inducing remodeling of necrotic myocardial area. Other therapeutic uses for the present invention include induction of skeletal muscle neogenesis and/or hyperplasia, kidney regeneration and/or for treatment of systemic and pulmonary hypertension.

Zpep14 induced coronary collateral development is measured in rabbits, dogs or pigs using models of chronic coronary occlusion (Landau et al., Amer. Heart J. 29:924-931, 1995; Sellke et al., Surgery 120(2):182-188, 1996; and Lazarous et al., 1996, ibid.) Zpep14 efficacy for treating stroke is tested in vivo, in rats, utilizing bilateral carotid artery occlusion and measuring histological changes, as well as maze performance (Gage et al., Neurobiol. Aging 9:645-655, 1988). Zpep14 efficacy in hypertension is tested in vivo utilizing spontaneously hypertensive rats (SHR) for systemic hypertension (Marche et al., Clin. Exp. Pharmacol. Physiol. Suppl. 1:S114-116, 1995). Moreover, other in vivo models for heart disease, such as the transgenic model for stunned myocardium may be employed to assay the effects zpep14 polypeptides on cardiac function (Murphy, A. M. et al., Science 287:488-491, 2000).

Proteins of the present invention are useful for example, in treating reproductive, prostate, testicular, uterine, stomach, heart, and other disorders, and can be measured in vitro using cultured cells or in vivo by administering molecules of the present invention to the appropriate animal model. For instance, host cells expressing a zpep14 polypeptide can be embedded in an alginate environment and injected (implanted) into recipient animals. Alginate-poly-L-lysine microencapsulation, permselective membrane encapsulation and diffusion chambers are a means to entrap transfected mammalian cells or primary mammalian cells. These types of non-immunogenic “encapsulations” permit the diffusion of proteins and other macromolecules secreted or released by the captured cells to the recipient animal. Most importantly, the capsules mask and shield the foreign, embedded cells from the recipient animal's immune response. Such encapsulations can extend the life of the injected cells from a few hours or days (naked cells) to several weeks (embedded cells). Alginate threads provide a simple and quick means for generating embedded cells.

The materials needed to generate the alginate threads are known in the art. In an exemplary procedure, 3% alginate is prepared in sterile H₂O, and sterile filtered. Just prior to preparation of alginate threads, the alginate solution is again filtered. An approximately 50% cell suspension (containing about 5×10⁵ to about 5×10⁷ cells/ml) is mixed with the 3% alginate solution. One ml of the alginate/cell suspension is extruded into a 100 mM sterile filtered CaCl₂ solution over a time period of ˜15 min, forming a “thread”. The extruded thread is then transferred into a solution of 50 mM CaCl₂, and then into a solution of 25 mM CaCl₂. The thread is then rinsed with deionized water before coating the thread by incubating in a 0.01% solution of poly-L-lysine. Finally, the thread is rinsed with Lactated Ringer's Solution and drawn from solution into a syringe barrel (without needle). A large bore needle is then attached to the syringe, and the thread is intraperitoneally injected into a recipient in a minimal volume of the Lactated Ringer's Solution.

An in vivo approach for assaying proteins of the present invention involves viral delivery systems. Exemplary viruses for this purpose include adenovirus, herpesvirus, retroviruses, vaccinia virus, and adeno-associated virus (AAV). Adenovirus, a double-stranded DNA virus, is currently the best studied gene transfer vector for delivery of heterologous nucleic acid (for review, see T. C. Becker et al., Meth. Cell Biol. 43:161-89, 1994; and J. T. Douglas and D. T. Curiel, Science & Medicine 4:44-53, 1997). The adenovirus system offers several advantages: (i) adenovirus can accommodate relatively large DNA inserts; (ii) can be grown to high-titer; (iii) infect a broad range of mammalian cell types; and (iv) can be used with many different promoters including ubiquitous, tissue specific, and regulatable promoters. Also, because adenoviruses are stable in the bloodstream, they can be administered by intravenous injection.

Using adenovirus vectors where portions of the adenovirus genome are deleted, inserts are incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

Moreover, adenoviral vectors containing various deletions of viral genes can be used in an attempt to reduce or eliminate immune responses to the vector. Such adenoviruses are E1 deleted, and in addition contain deletions of E2A or E4 (Lusky, M. et al., J. Virol. 72:2022-2032, 1998; Raper, S. E. et al., Human Gene Therapy 9:671-679, 1998). In addition, deletion of E2b is reported to reduce immune responses (Amalfitano, A. et al., J. Virol. 72:926-933, 1998). Moreover, by deleting the entire adenovirus genome, very large inserts of heterologous DNA can be accommodated. Generation of so called “gutless” adenoviruses where all viral genes are deleted are particularly advantageous for insertion of large inserts of heterologous DNA. For review, see Yeh, P. and Perricaudet, M., FASEB J. 11:615-623, 1997.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293 cells can be grown as adherent cells or in suspension culture at relatively high cell density to produce significant amounts of protein (See Gamier et al., Cytotechnol. 15:145-55, 1994). With either protocol, an expressed, secreted heterologous protein can be repeatedly isolated from the cell culture supernatant, lysate, or membrane fractions depending on the disposition of the expressed protein in the cell. Within the infected 293 cell production protocol, non-secreted proteins may also be effectively obtained.

As a ligand, the activity of zpep14 polypeptide can be measured by a silicon-based biosensor microphysiometer which measures the extracellular acidification rate or proton excretion associated with receptor binding and subsequent physiologic cellular responses. An exemplary device is the Cytosensor™ Microphysiometer manufactured by Molecular Devices, Sunnyvale, Calif. A variety of cellular responses, such as cell proliferation, ion transport, energy production, inflammatory response, regulatory and receptor activation, and the like, can be measured by this method. See, for example, McConnell, H. M. et al., Science 257:1906-1912, 1992; Pitchford, S. et al., Meth. Enzymol. 228:84-108, 1997; Arimilli, S. et al., J. Immunol. Meth. 212:49-59, 1998; Van Liefde, I. et al., Eur. J. Pharmacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli, including zpep14 polypeptide, its agonists, or antagonists. Preferably, the microphysiometer is used to measure responses of a zpep14-responsive eukaryotic cell, compared to a control eukaryotic cell that does not respond to zpep14 polypeptide. Zpep14-responsive eukaryotic cells comprise cells into which a receptor for zpep14 has been transfected creating a cell that is responsive to zpep14; or cells naturally responsive to zpep14 such as cells derived from prostate, testis, uterine tissue, or the like. Differences, measured by a change, for example, an increase or diminution in extracellular acidification, in the response of cells exposed to zpep14 polypeptide, relative to a control not exposed to zpep14, are a direct measurement of zpep14-modulated cellular responses. Moreover, such zpep14-modulated responses can be assayed under a variety of stimuli. Using the microphysiometer, there is provided a method of identifying agonists of zpep14 polypeptide, comprising providing cells responsive to a zpep14 polypeptide, culturing a first portion of the cells in the absence of a test compound, culturing a second portion of the cells in the presence of a test compound, and detecting a change, for example, an increase or diminution, in a cellular response of the second portion of the cells as compared to the first portion of the cells. The change in cellular response is shown as a measurable change extracellular acidification rate. Moreover, culturing a third portion of the cells in the presence of zpep14 polypeptide and the absence of a test compound can be used as a positive control for the zpep14-responsive cells, and as a control to compare the agonist activity of a test compound with that of the zpep14 polypeptide. Moreover, using the microphysiometer, there is provided a method of identifying antagonists of zpep14 polypeptide, comprising providing cells responsive to a zpep14 polypeptide, culturing a first portion of the cells in the presence of zpep14 and the absence of a test compound, culturing a second portion of the cells in the presence of zpep14 and the presence of a test compound, and detecting a change, for example, an increase or a diminution in a cellular response of the second portion of the cells as compared to the first portion of the cells. The change in cellular response is shown as a measurable change extracellular acidification rate. Antagonists and agonists, for zpep14 polypeptide, can be rapidly identified using this method.

Moreover, zpep14 can be used to identify cells, tissues, or cell lines which respond to a zpep14-stimulated pathway. The microphysiometer, described above, can be used to rapidly identify ligand-responsive cells, such as cells responsive to zpep14 of the present invention. Cells can be cultured in the presence or absence of zpep14 polypeptide. Those cells which elicit a measurable change in extracellular acidification in the presence of zpep14 are responsive to zpep14. Such cell lines, can be used to identify antagonists and agonists of zpep14 polypeptide as described above.

In view of the tissue distribution observed for zpep14 polypeptides, agonists (including the natural ligand/substrate/cofactor/etc.) and antagonists have enormous potential in both in vitro and in vivo applications. For example, zpep14 polypeptide and agonist compounds are useful as components of defined cell culture media, and may be used alone or in combination with cytokines and hormones to replace serum that is commonly used in cell culture. Agonists are thus useful in specifically promoting the growth and/or development of mammalian cells in vitro, particularly of those derived from reproductive tissues. As such, zpep14 polypeptides or agonists are added to tissue culture media for these cell types.

Zpep14 can also be used to identify inhibitors (antagonists) of its activity. Test compounds are added to assays disclosed herein to identify compounds that inhibit the activity of zpep14. In addition to those assays disclosed herein, samples can be tested for inhibition of zpep14 activity within a variety of assays designed to measure receptor binding or the stimulation/inhibition of zpep14-dependent cellular responses. For example, zpep14-responsive cell lines can be transfected with a reporter gene construct that is responsive to a zpep14-stimulated cellular pathway. Reporter gene constructs of this type are known in the art, and will generally comprise a zpep14-DNA response element operably linked to a gene encoding an assayable protein, such as luciferase. DNA response elements can include, but are not limited to, cyclic AMP response elements (CRE), hormone response elements (HRE) insulin response element (IRE) (Nasrin et al., Proc. Natl. Acad. Sci. USA 87:5273-7, 1990) and serum response elements (SRE) (Shaw et al. Cell 56: 563-72, 1989). Cyclic AMP response elements are reviewed in Roestler et al., J. Biol. Chem. 263 (19):9063-6; 1988 and Habener, Molec. Endocrinol. 4 (8):1087-94; 1990. Hormone response elements are reviewed in Beato, Cell 56:335-44; 1989. Candidate compounds, solutions, mixtures or extracts are tested for the ability to inhibit the activity of zpep14 on the target cells as evidenced by a decrease in zpep14 stimulation of reporter gene expression. Assays of this type will detect compounds that directly block zpep14 binding to cell-surface receptors, as well as compounds that block processes in the cellular pathway subsequent to receptor-ligand binding. In the alternative, compounds or other samples can be tested for direct blocking of zpep14 binding to receptor using zpep14 tagged with a detectable label (e.g., ¹²⁵I, biotin, horseradish peroxidase, FITC, and the like). Within assays of this type, the ability of a test sample to inhibit the binding of labeled zpep14 to the receptor is indicative of inhibitory activity, which can be confirmed through secondary assays. Receptors used within binding assays may be cellular receptors or isolated, immobilized receptors.

The tissue specificity of zpep14 expression suggests a role in spermatogenesis, a process that is remarkably similar to the development of blood cells (hematopoiesis). Briefly, spermatogonia undergo a maturation process similar to the differentiation of hematopoietic stem cells. In view of the tissue specificity observed for zpep14, agonists and antagonists have enormous potential in both in vitro and in vivo applications. Zpep14 polypeptides, agonists and antagonists may also prove useful in modulating spermatogenesis and thus aid in overcoming infertility. Antagonists are useful as research reagents for characterizing sites of ligand-receptor interaction. In vivo, zpep14 polypeptides, agonists or antagonists may find application in the treatment of male infertility or as a male contraceptive agents.

The zpep14 polypeptides, antagonists of agonists, of the present invention can also modulate sperm capacitation. Before reaching the oocyte or egg and initiating an egg-sperm interaction, the sperm must be activated. The sperm undergo a gradual capacitation, lasting up to 3 or 4 hours in vitro, during which the plasma membrane of the sperm head and the outer acrosomal membrane fuse to form vesicles that facilitate the release of acrosomal enzymes. The acrosomal membrane surrounds the acrosome or acrosomal cap which is located at the anterior end of the nucleus in the sperm head. In order for the sperm to fertilize egg the sperm must penetrate the oocyte. To enable this process the sperm must undergo acrosomal exocytosis, also known as the acrosomal reaction, and release the acrosomal enzymes in the vicinity of the oocyte. These enzymes enable the sperm to penetrate the various oocyte layers, (the cumulus oophorus, the corona radiata and the zona pellucida). The released acrosomal enzymes include hyaluronidase and proacrosin, in addition to other enzymes such as proteases. During the acrosomal reaction, proacrosin is converted to acrosin, the active form of the enzyme, which is required for and must occur before binding and penetration of the zona pellucida is possible. A combination of the acrosomal lytic enzymes and sperm tail movements allow the sperm to penetrate the oocyte layers. Numerous sperm must reach the egg and release acrosomal enzymes before the egg can finally be fertilized. Only one sperm will successfully bind to, penetrate and fertilize the egg, after which the zona hardens so that no other sperm can penetrate the egg (Zaneveld, in Male Infertility Chapter 11, Comhaire (Ed.), Chapman & Hall, London, 1996). Peptide hormones, such as insulin homologs are associated with sperm activation and egg-sperm interaction. For instance, capacitated sperm incubated with relaxin show an increased percentage of progressively motile sperm, increased zona penetration rates, and increased percentage of viable acrosome-reacted sperm (Carrell et al., Endocr. Res. 21:697-707, 1995). Similarity of the zpep14 polypeptide structure with peptide hormones and localization of Zpep14 to the testis, prostate and uterus suggests that the zpep14 polypeptides described herein play a role in these and other reproductive processes.

Accordingly, proteins of the present invention can have applications in enhancing fertilization during assisted reproduction in humans and in animals. Such assisted reproduction methods are known in the art and include artificial insemination, in vitro fertilization, embryo transfer and gamete intrafallopian transfer. Such methods are useful for assisting men and women who have physiological or metabolic disorders preventing natural conception or can be used to enhance in vitro fertilization. Such methods are also used in animal breeding programs, such as for livestock breeding and could be used as methods for the creation of transgenic animals. Proteins of the present invention can be combined with sperm, an egg or an egg-sperm mixture prior to fertilization of the egg. In some species, sperm capacitate spontaneously during in vitro fertilization procedures, but normally sperm capacitate over an extended period of time both in vivo and in vitro. It is advantageous to increase sperm activation during such procedures to enhance the likelihood of successful fertilization. The washed sperm or sperm removed from the seminal plasma used in such assisted reproduction methods has been shown to have altered reproductive functions, in particular, reduced motility and zona interaction. To enhance fertilization during assisted reproduction methods sperm is capacitated using exogenously added compounds. Suspension of the sperm in seminal plasma from normal subjects or in a “capacitation media” containing a cocktail of compounds known to activate sperm, such as caffeine, dibutyl cyclic adenosine monophosphate (dbcAMP) or theophylline, have resulted in improved reproductive function of the sperm, in particular, sperm motility and zonae penetration (Park et al., Am. J. Obstet. Gynecol. 158:974-9, 1988; Vandevoort et al., Mol. Repro. Develop. 37:299-304, 1993; Vandevoort and Overstreet, J. Androl. 16:327-33, 1995). The presence of immunoreactive relaxin in vivo and in association with cryopreserved semen, was shown to significantly increase sperm motility (Juang et al., Anim. Reprod. Sci. 20:21-9, 1989; Juang et al., Anim. Reprod. Sci. 22:47-53, 1990). Porcine relaxin stimulated sperm motility in cryopreserved human sperm (Colon et al., Fertil. Steril. 46:1133-39, 1986; Lessing et al., Fertil. Steril. 44:406-9, 1985) and preserved ability of washed human sperm to penetrate cervical mucus in vitro (Brenner et al., Fertil. Steril. 42:92-6, 1984). Polypeptides of the present invention can used in such methods to enhance viability of cryopreserved sperm, enhance sperm motility and enhance fertilization, particularly in association with methods of assisted reproduction.

In cases where pregnancy is not desired, zpep14 polypeptide or polypeptide fragments may function as germ-cell-specific antigens for use as components in “immunocontraceptive” or “anti-fertility” vaccines to induce formation of antibodies and/or cell mediated immunity to selectively inhibit a process, or processes, critical to successful reproduction in humans and animals. The use of sperm and testis antigens in the development of immunocontraceptives have been described (O'Hern et al., Biol Reprod. 52:311-39, 1995; Diekman and Herr, Am. J. Reprod. Immunol. 37:111-17, 1997; Zhu and Naz, Proc. Natl. Acad. Sci. USA 94:4704-9,1997). A vaccine based on human chorionic gonadotrophin (HCG) linked to a diphtheria or tetanus carrier was in clinical trials (Talwar et al., Proc. Natl. Acad. Sci. USA 91:8532-36, 1994). A single injection resulted in production of high titer antibodies that persisted for nearly a year in rabbits (Stevens, Am. J. Reprod. Immunol. 29:176-88, 1993). Such methods of immunocontraception using vaccines would include a zpep14 testes-specific protein or fragment thereof. The Zpep14 protein or fragments can be conjugated to a carrier protein or peptide, such as tetanus or diphtheria toxoid. An adjuvant, as described above, can be included and the protein or fragment can be noncovalently associated with other molecules to enhance intrinsic immunoreactivity. Methods for administration and methods for determining the number of administrations are known in the art. Such a method might include a number of primary injections over several weeks followed by booster injections as needed to maintain a suitable antibody titer.

Regulation of reproductive function in males and females is controlled in part by feedback inhibition of the hypothalamus and anterior pituitary by blood-borne hormones. Testis proteins, such as activins and inhibins, have been shown to regulate secretion of active molecules including follicle stimulating hormone (FSH) from the pituitary (Ying, Endodcr. Rev. 9:267-93, 1988; Plant et al., Hum. Reprod. 8:41-44,1993). Inhibins, also expressed in the ovaries, have been shown to regulate ovarian functions (Woodruff et al., Endocr. 132:2332-42,1993; Russell et al., J. Reprod. Fertil. 100:115-22, 1994). Relaxin has been shown to be a systemic and local acting hormone regulating follicular and uterine growth (Bagnell et al., J. Reprod. Fertil. 48:127-38, 1993). As such, the polypeptides of the present invention may also have effects on female gametes and reproductive tract. These functions may also be associated with zpep14 polypeptides and may be used to regulate testicular or ovarian functions.

A zpep14 polypeptide can be expressed as a fusion with an immunoglobulin heavy chain constant region, typically an F_{c} fragment, which contains two constant region domains and lacks the variable region. Methods for preparing such fusions are disclosed in U.S. Pat. Nos. 5,155,027 and 5,567,584. Such fusions are typically secreted as multimeric molecules wherein the Fc portions are disulfide bonded to each other and two non-Ig polypeptides are arrayed in closed proximity to each other. Fusions of this type can be used as drug-delivery devices, to stimulate a zpep14-induced signal transduction cascade in vivo or in vitro, or to affinity purify zpep14 receptors, as in vitro assay tool, or as an antagonist. For use in assays, the chimeras are bound to a support via the F_{c} region and used in an ELISA format.

A zpep14 ligand-binding polypeptide can also be used for purification of ligand. The polypeptide is immobilized on a solid support, such as agarose beads, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids containing ligand are passed through the column one or more times to allow ligand to bind to the receptor polypeptide. The ligand is then eluted using changes in salt concentration, chaotropic agents (guanidine HCl), or pH to disrupt ligand-receptor binding.

An assay system that uses a ligand-binding receptor (or an antibody, one member of a complement/anti-complement pair) or a binding fragment thereof, and a commercially available biosensor instrument (BIAcore, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding.

Ligand-binding receptor polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

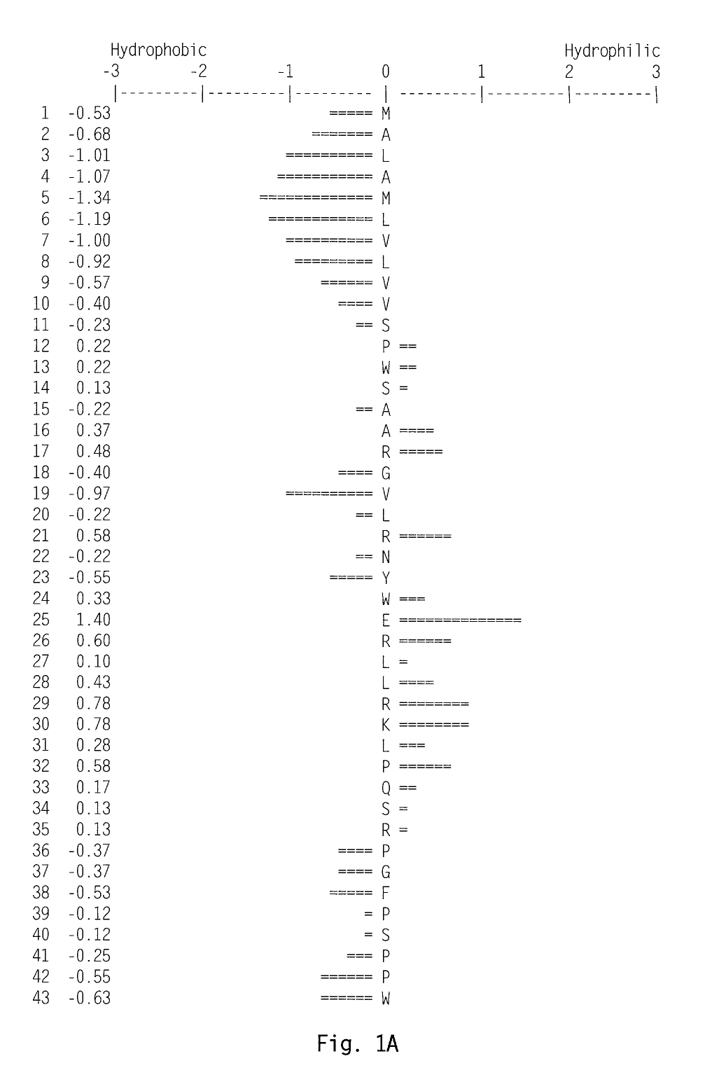

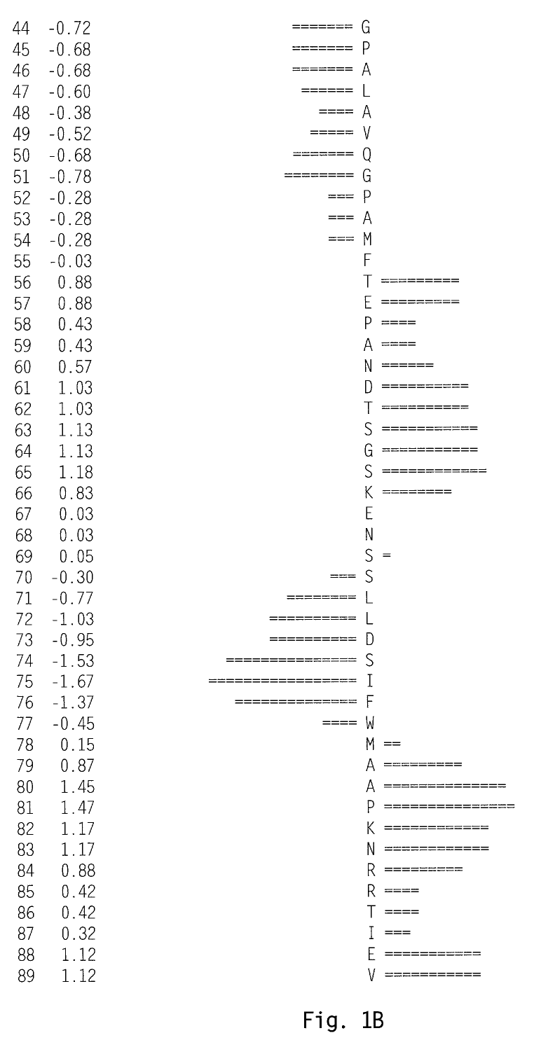

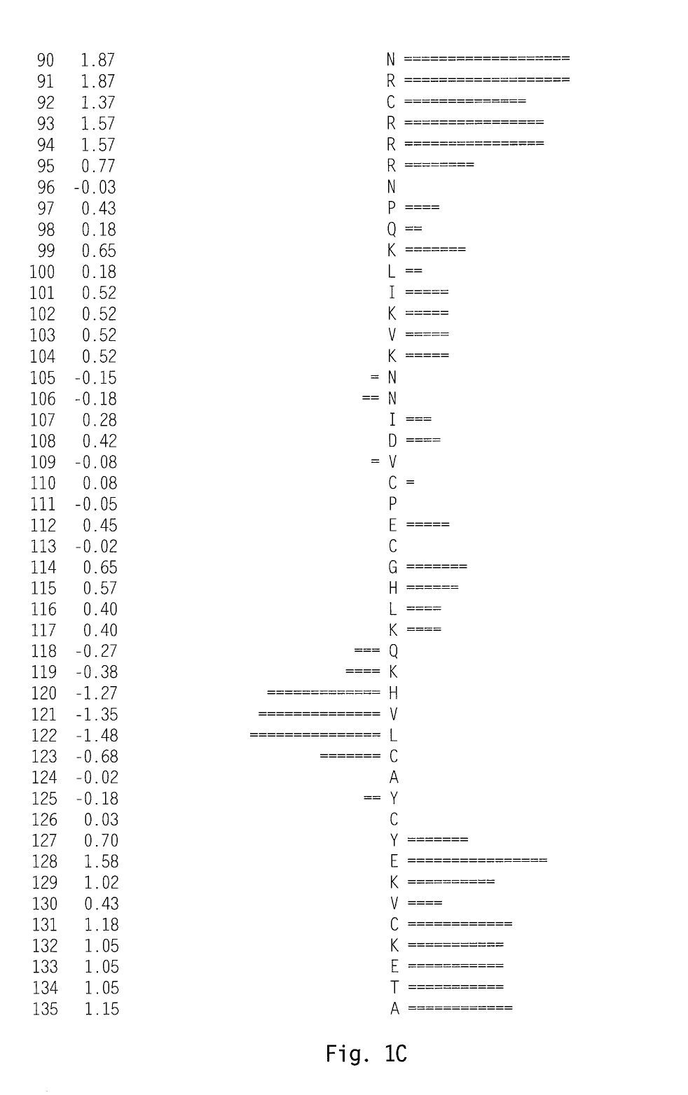

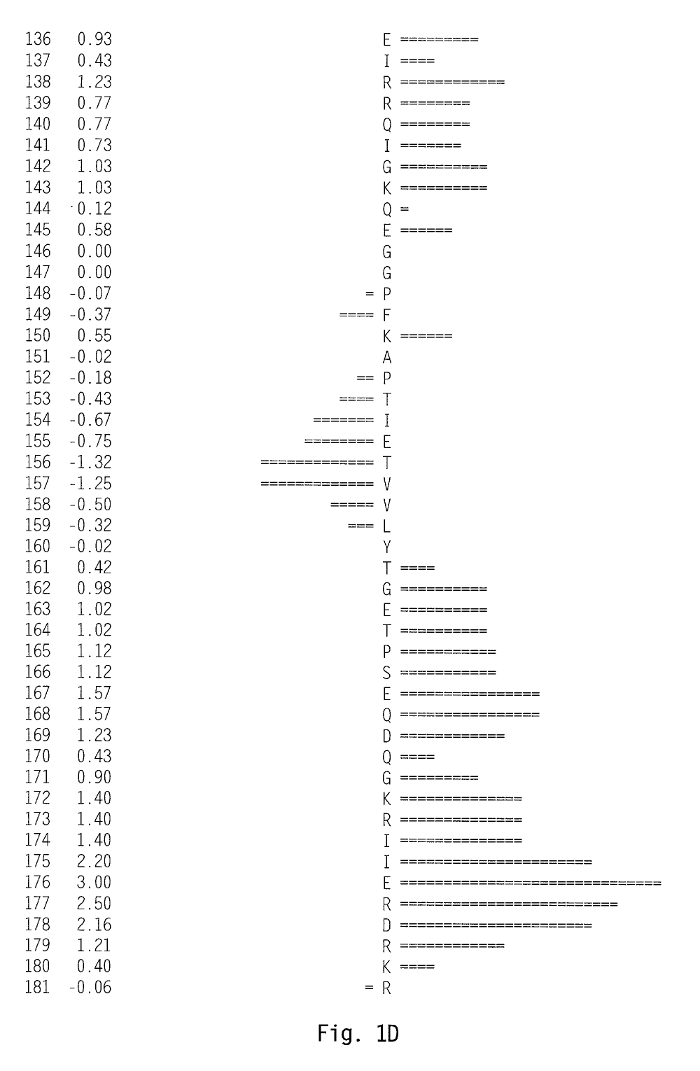

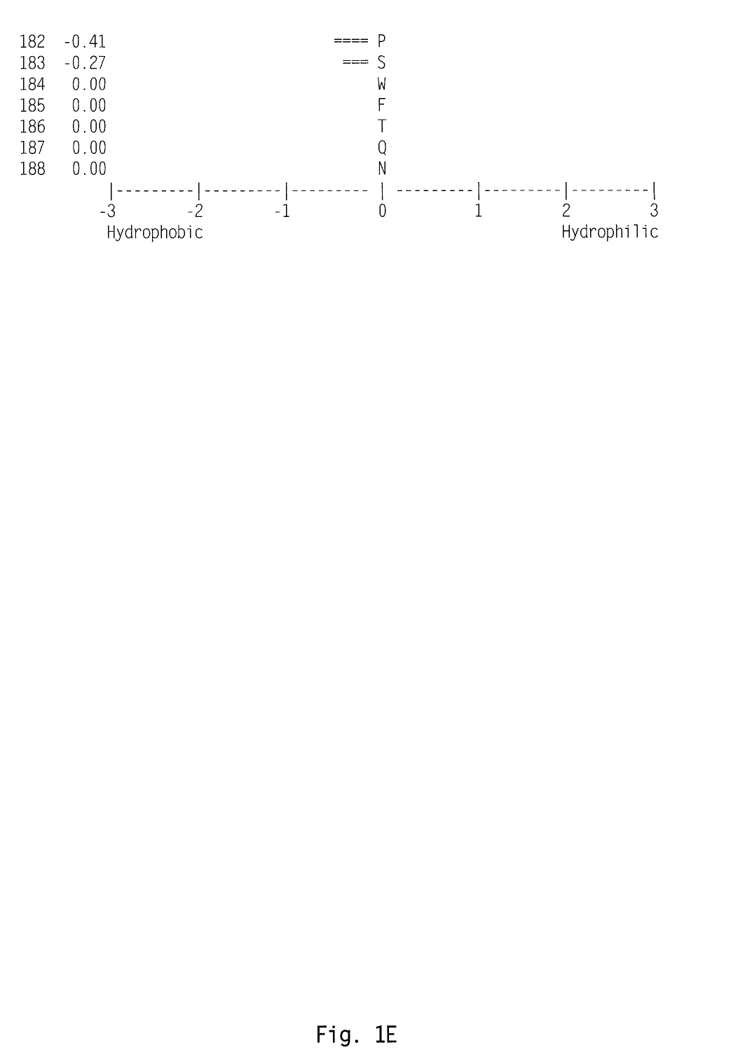

Zpep14 polypeptides can also be used to prepare antibodies that bind to zpep14 epitopes, peptides or polypeptides. The zpep14 polypeptide or a fragment thereof serves as an antigen (immunogen) to inoculate an animal and elicit an immune response. One of skill in the art would recognize that antigenic, epitope-bearing polypeptides contain a sequence of at least 6, preferably at least 9, and more preferably at least 15 to about 30 contiguous amino acid residues of a zpep14 polypeptide (e.g., SEQ ID NO:2). Polypeptides comprising a larger portion of a zpep14 polypeptide, i.e., from 10 to 30 residues up to the entire length of the amino acid sequence are included. Antigens or immunogenic epitopes can also include attached tags, adjuvants and carriers, as described herein. Suitable antigens include the zpep14 polypeptide encoded by SEQ ID NO:2 from amino acid number 17 (Arg) to amino acid number 188 (Asn), or a contiguous 9 to 172 amino acid fragment thereof. Other suitable antigens include polypeptides-1 through -9, disclosed herein. Preferred peptides to use as antigens are hydrophilic peptides such as those predicted by one of skill in the art from a hydrophobicity plot (See FIG. 1). Zpep14 hydrophilic peptides include peptides comprising amino acid sequences selected from the group consisting of: (1) amino acid number 90 (Asn) to amino acid number 95 (Arg) of SEQ ID NO:2; (2) amino acid number 128 (Glu) to amino acid number 133 (Glu) of SEQ ID NO:2; (3) amino acid number 167 (Glu) to amino acid number 172 (Lys) of SEQ ID NO:2; (4) amino acid number 175 (Ile) to amino acid number 180 (Lys) of SEQ ID NO:2; and (5) amino acid number 176 (Glu) to amino acid number 181 (Arg) of SEQ ID NO:2. Antibodies from an immune response generated by inoculation of an animal with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, and rats with a zpep14 polypeptide or a fragment thereof. The immunogenicity of a zpep14 polypeptide may be increased through the use of an adjuvant, such as alum (aluminum hydroxide) or Freund's complete or incomplete adjuvant. Polypeptides useful for immunization also include fusion polypeptides, such as fusions of zpep14 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced.

Alternative techniques for generating or selecting antibodies useful herein include in vitro exposure of lymphocytes to zpep14 protein or peptide, and selection of antibody display libraries in phage or similar vectors (for instance, through use of immobilized or labeled zpep14 protein or peptide). Genes encoding polypeptides having potential zpep14 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the zpep14 sequences disclosed herein to identify proteins which bind to zpep14. These “binding polypeptides” which interact with zpep14 polypeptides can be used for tagging cells; for isolating homolog polypeptides by affinity purification; they can be directly or indirectly conjugated to drugs, toxins, radionuclides and the like. These binding polypeptides can also be used in analytical methods such as for screening expression libraries and neutralizing activity, e.g., for blocking interaction between ligand and receptor, or viral binding to a receptor. The binding polypeptides can also be used for diagnostic assays for determining circulating levels of zpep14 polypeptides; for detecting or quantitating soluble zpep14 polypeptides as marker of underlying pathology or disease. These binding polypeptides can also act as zpep14 “antagonists” to block zpep14 binding and signal transduction in vitro and in vivo. These anti-zpep14 binding polypeptides would be useful for inhibiting zpep14 activity or protein-binding.

Antibodies are considered to be specifically binding if: 1) they exhibit a threshold level of binding activity, and 2) they do not significantly cross-react with related polypeptide molecules. A threshold level of binding is determined if anti-zpep14 antibodies herein bind to a zpep14 polypeptide, peptide or epitope with an affinity at least 10-fold greater than the binding affinity to control (non-zpep14) polypeptide. It is preferred that the antibodies exhibit a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, G., Ann. NY Acad. Sci. 51: 660-672, 1949).

Whether anti-zpep14 antibodies do not significantly cross-react with related polypeptide molecules is shown, for example, by the antibody detecting zpep14 polypeptide but not known related polypeptides using a standard Western blot analysis (Ausubel et al., ibid.). Examples of known related polypeptides are those disclosed in the prior art, such as known orthologs, and paralogs, and similar known members of a protein family, Screening can also be done using non-human zpep14, and zpep14 mutant polypeptides. Moreover, antibodies can be “screened against” known related polypeptides, to isolate a population that specifically binds to the zpep14 polypeptides. For example, antibodies raised to zpep14 are adsorbed to related polypeptides adhered to insoluble matrix; antibodies specific to zpep14 will flow through the matrix under the proper buffer conditions. Screening allows isolation of polyclonal and monoclonal antibodies non-crossreactive to known closely related polypeptides (Antibodies: A Laboratory Manual, Harlow and Lane (eds.), Cold Spring Harbor Laboratory Press, 1988; Current Protocols in Immunology, Cooligan, et al. (eds.), National Institutes of Health, John Wiley and Sons, Inc., 1995). Screening and isolation of specific antibodies is well known in the art. See, Fundamental Immunology, Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984. Specifically binding anti-zpep14 antibodies can be detected by a number of methods in the art, and disclosed below.

A variety of assays known to those skilled in the art can be utilized to detect antibodies which bind to zpep14 proteins or polypeptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant zpep14 protein or polypeptide.

Antibodies to zpep14 may be used for tagging cells that express zpep14; for isolating zpep14 by affinity purification; for diagnostic assays for determining circulating levels of zpep14 polypeptides; for detecting or quantitating soluble zpep14 as marker of underlying pathology or disease; in analytical methods employing FACS; for screening expression libraries; for generating anti-idiotypic antibodies; and as neutralizing antibodies or as antagonists to block zpep14 activity in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to zpep14 or fragments thereof may be used in vitro to detect denatured zpep14 or fragments thereof in assays, for example, Western Blots or other assays known in the art.

Antibodies or polypeptides herein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (receptor or antigen, respectively, for instance). More specifically, zpep14 polypeptides or anti-zpep14 antibodies, or bioactive fragments or portions thereof, can be coupled to detectable or cytotoxic molecules and delivered to a maimal having cells, tissues or organs that express the anti-complementary molecule.

Suitable detectable molecules may be directly or indirectly attached to the polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Polypeptides or antibodies may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the polypeptide or antibody portion. For these purposes, biotin/streptavidin is an exemplary complementary/anticomplementary pair.

In another embodiment, polypeptide-toxin fusion proteins or antibody-toxin fusion proteins can be used for targeted cell or tissue inhibition or ablation (for instance, to treat cancer cells or tissues). Alternatively, if the polypeptide has multiple functional domains (i.e., an activation domain or a receptior binding domain, plus a targeting domain), a fusion protein including only the targeting domain may be suitable for directing a detectable molecule, a cytotoxic molecule or a complementary molecule to a cell or tissue type of interest. In instances where the domain only fusion protein includes a complementary molecule, the anti-complementary molecule can be conjugated to a detectable or cytotoxic molecule. Such domain-complementary molecule fusion proteins thus represent a generic targeting vehicle for cell/tissue-specific delivery of generic anti-complementary-detectable/cytotoxic molecule conjugates.

In another embodiment, zpep14-cytokine fusion proteins or antibody-cytokine fusion proteins can be used for enhancing in vivo killing of target tissues (for example, blood and bone marrow cancers), if the zpep14 polypeptide or anti-zpep14 antibody targets the hyperproliferative blood or bone marrow cell (See, generally, Hornick et al., Blood 89:4437-47, 1997). Hornick et al. described fusion proteins that target a cytokine to a desired site of action, thereby providing an elevated local concentration of cytokine. Suitable zpep14 polypeptides or anti-zpep14 antibodies can target an undesirable cell or tissue (i.e., a tumor or a leukemia), and the fused cytokine can mediate improved target cell lysis by effector cells. Suitable cytokines for this purpose include interleukin 2 and granulocyte-macrophage colony-stimulating factor (GM-CSF), for instance.

In yet another embodiment, if the zpep14 polypeptide or anti-zpep14 antibody targets vascular cells or tissues, such polypeptide or antibody may be conjugated with a radionuclide, and particularly with a beta-emitting radionuclide, to reduce restenosis. Such therapeutic approach poses less danger to clinicians who administer the radioactive therapy. For instance, iridium-192 impregnated ribbons placed into stented vessels of patients until the required radiation dose was delivered showed decreased tissue growth in the vessel and greater luminal diameter than the control group, which received placebo ribbons. Further, revascularisation and stent thrombosis were significantly lower in the treatment group. Similar results are predicted with targeting of a bioactive conjugate containing a radionuclide, as described herein.

The bioactive polypeptide or antibody conjugates described herein can be delivered intravenously, intraarterially or intraductally, or may be introduced locally at the intended site of action.

Molecules of the present invention can be used to identify and isolate receptors that bind zpep14 polypeptide. For example, proteins and peptides of the present invention can be immobilized on a column and membrane preparations run over the column (Immobilized Affinity Ligand Techniques, Hermanson et al., eds., Academic Press, San Diego, Calif., 1992, pp.195-202). Proteins and peptides can also be radiolabeled (Methods in Enzymol., vol. 182, “Guide to Protein Purification”, M. Deutscher, ed., Acad. Press, San Diego, 1990, 721-37) or photoaffinity labeled (Brunner et al., Ann. Rev. Biochem. 62:483-514, 1993 and Fedan et al., Biochem. Pharmacol. 33:1167-80, 1984) and specific cell-surface proteins can be identified.

The polypeptides, antagonists, agonists, nucleic acid and/or antibodies of the present invention may be used in treatment of disorders associated with gonadal development, pregnancy, pubertal changes, menopause, ovarian cancer, fertility, ovarian function, polycystic ovarian syndrome, uterine cancer, endometriosis, libido, mylagia and neuralgia associated with reproductive phenomena, male sexual dysfunction, impotency, prostate cancer, testicular cancer, stomach cancer, gastrointestinal mobility and dysfunction. The molecules of the present invention may used to modulate or to treat or prevent development of pathological conditions in such diverse tissue as prostate and uterus. In particular, certain syndromes or diseases may be amenable to such diagnosis, treatment or prevention. Moreover, natural functions, such as embryo implantation or spermatogenesis, may be suppressed or controlled for use in birth control by molecules of the present invention.

Zpep14 polypeptide is expressed in the uterus and may have additional biological activity independent of prostate or testis function, as described herein. Oogenesis is the process by which a diploid stem cell proceeds through multiple stages of differentiation, culminating in the formation of a terminally differentiated cell with a unique function, an oocyte. Unlike spermatogenesis, which begins at puberty and continues on through the life of a male, oogenesis begins during fetal development and by birth, a female's entire supply of primary oocytes are stored in the ovaries in primordial follicles and await maturation and release.

In the adult ovary, folliculogenesis starts when the follicles enter the growth phase. Early growing follicles undergo a dramatic process of cellular proliferation and differentiation. The classic control of ovarian function by luteinizing hormone (LH) and follicle stimulating hormone (FSH) is now thought to include the action of a variety of molecules that act to promote cell-cell interactions between cells of the follicle. For review, see Gougeon, A., Endocrine Rev. 17:121-155, 1996. Hence, the mechanisms for controlling ovarian folliculogenesis and dominant follicle selection are still under investigation. As zpep14 is expressed in the uterus, it may serve a role in modulating ovarian function by regulating folliculogenesis and dominant follicle selection, by affecting proliferation or differentiation of follicular cells, affecting cell-cell interactions, modulating hormones involved in the process, and the like.

The ovarian cycle in mammals includes the growth and maturation of follicles, followed by ovulation and transformation of follicles into corpea lutea. The physiological events in the ovarian cycle are dependent on interactions between hormones and cells within the hypothalamic-pituitary-ovarian axis, including gonadotropin releasing hormone (GnRH), LH, and FSH. In addition, estradiol, synthesized in the follicle, primes the hypothalamic-pituitary axis and is required for the mid-cycle surge of gonadotropin that stimulates the resumption of oocyte meiosis and leads to ovulation and subsequent extrusion of an oocyte from the follicle. This gonadotropin surge also promotes the differentiation of the follicular cells from secreting estradiol to secreting progesterone. Progesterone, secreted by the corpus luteum, is needed for uterine development required for the implantation of fertilized oocytes. The central role of hypothalamic-pituitary-gonadal hormones in the ovarian cycle and reproductive cascade, and the role of sex steroids on target tissues and organs, e.g., uterus, breast, adipose, bones and liver, has made modulators of their activity desirable for therapeutic applications. Such applications include treatments for precocious puberty, endometriosis, uterine leiomyomata, hirsutism, infertility, pre menstrual syndrome (PMS), amenorrhea, and as contraceptive agents.

Zpep14 polypeptides, agonists and antagonists which modulate the actions of such hormones can be of therapeutic value. Such molecules can also be useful for modulating steroidogenesis, both in vivo and in vitro, and modulating aspects of the ovarian cycle such as oocyte maturation, ovarian cell-cell interactions, follicular development and rupture, luteal function, menstruation, and promoting uterine implantation of fertilized oocytes. Molecules which modulate hormone action can be beneficial therapeutics for use prior to or at onset of puberty, or in adult women. For example, puberty in females is marked by an establishment of feed-back loops to control hormone levels and hormone production. Abnormalities resulting from hormone imbalances during puberty have been observed and include precocious puberty, where pubertal changes occur in females prior to the age of 8. Hormone-modulating molecules, can be used, in this case, to suppress hormone secretion and delay onset of puberty.

The level and ratio of gonadotropin and steroid hormones can be used to assess the existence of hormonal imbalances associated with diseases, as well as determine whether normal hormonal balance has been restored after administration of a therapeutic agent. Determination of estradiol, progesterone, LH, and FSH, for example, from serum is known by one of skill in the art. Such assays can be used to monitor the hormone levels after administration of zpep14 in vivo, or in a transgenic mouse model where the zpep14 gene is expressed or the murine ortholog is deleted. Thus, as a hormone-modulating molecule, zpep14 polypeptides can have therapeutic application for treating, for example, breakthrough menopausal bleeding, as part of a therapeutic regime for pregnancy support, or for treating symptoms associated with polycystic ovarian syndrome (PCOS), endometriosis, PMS and menopause. In addition, other in vivo rodent models are known in the art to assay effects of zpep14 polypeptide on, for example, polycystic ovarian syndrome (PCOS).

Proteins of the present invention may also be used in applications for enhancing fertilization during assisted reproduction in humans and in animals. Such assisted reproduction methods are known in the art and include artificial insemination, in vitro fertilization, embryo transfer, and gamete intrafallopian transfer. Such methods are useful for assisting those who may have physiological or metabolic disorders that prevent or impede natural conception. Such methods are also used in animal breeding programs, e.g., for livestock, racehorses, domestic and wild animals, and could be used as methods for the creation of transgenic animals. Zpep14 polypeptides could be used in the induction of ovulation, either independently or in conjunction with a regimen of gonadotropins or agents such as clomiphene citrate or bromocriptine (Speroff et al., Induction of ovulation, Clinical Gynecologic Endocrinology and Infertility, 5^{th} ed., Baltimore, Williams & Wilkins, 1994). As such, proteins of the present invention can be administered to the recipient prior to fertilization or combined with the sperm, an egg or an egg-sperm mixture prior to in vitro or in vivo fertilization. Such proteins can also be mixed with oocytes prior to cryopreservation to enhance viability of the preserved oocytes for use in assisted reproduction.

The zpep14 polypeptides, agonists and antagonists of the present invention may be directly used as or incorporated into therapies for treating reproductive disorders. Disorders such as luteal phase deficiency would benefit from such therapy (Soules, “Luteal phase deficiency: A subtle abnormality of ovulation” in, Infertility: Evaluation and Treatment, Keye et al., eds., Philadelphia, W B Saunders, 1995). Moreover, administration of gonadotropin-releasing hormone is shown to stimulate reproductive behavior (Riskin and Moss, Res. Bull. 11:481-5, 1983; Kadar et al., Physiol. Behav. 51:601-5, 1992 and Silver et al., J. Neruoendocrin. 4:207-10, 199; King and Millar, Cell. Mol. Neurobiol., 15:5-23, 1995). Given the high prevalence of sexual dysfunction and impotence in humans, molecules, such as zpep14, which may modulate or enhance gonadotropin activity can find application in developing treatments for these conditions. Conversely, polypeptides of the present invention, their antagonists or agonists can be used to inhibit normal reproduction in the form of birth control, for example, by decreasing spermatogenesis or preventing uterine implantation of a fertilized egg.

The zpep14 polypeptides of the present invention can be used to study ovarian cell proliferation, maturation, and differentiation, i.e., by acting as a luteinizing agent that converts granulosa cells from estradiol to progesterone-producing cells. Such methods of the present invention generally comprise incubating granulosa cells, theca cells, oocytes or a combination thereof, in the presence and absence of zpep14 polypeptide, monoclonal antibody, agonist or antagonist thereof and observing changes in cell proliferation, maturation and differentiation. See for example, Basini et al., (J. Rep. Immunol. 37:139-53, 1998); Duleba et al., (Fert. Ster. 69:335-40, 1998); and Campbell, B. K. et al., J. Reprod. and Fert. 112:69-77, 1998).

The polypeptides, antagonists, agonists, nucleic acid and/or antibodies of the present invention can also be used in treatment of disorders associated with gastrointestinal cell contractility, secretion of digestive enzymes and acids, gastrointestinal motility, recruitment of digestive enzymes; inflammation, particularly as it affects the gastrointestinal system; reflux disease and regulation of nutrient absorption. Specific conditions that will benefit from treatment with molecules of the present invention include, but are not limited to, diabetic gastroparesis, post-surgical gastroparesis, vagotomy, chronic idiopathic intestinal pseudo-obstruction and gastroesophageal reflux disease. Additional uses include, gastric emptying for radiological studies, stimulating gallbladder contraction and antrectomy.

The motor and neurological affects of molecules of the present invention make it useful for treatment of obesity and other metabolic disorders where neurological feedback modulates nutritional absorption. The molecules of the present invention are useful for regulating satiety, glucose absorption and metabolism, and neuropathy-associated gastrointestinal disorders. Molecules of the present invention are also useful as additives to anti-hypoglycemic preparations containing glucose and as adsorption enhancers for oral drugs which require fast nutrient action. Additionally, molecules of the present invention can be used to stimulate glucose-induced insulin release.

Moreover, tissues in which the polypeptides of the present invention are expressed are comprised in part of epithelial cells where zpep14 polypeptides, agonists or antagonists thereof may be therapeutically useful for promoting wound healing. To verify the presence of this capability in zpep14 polypeptides, agonists or antagonists of the present invention, such zpep14 polypeptides, agonists or antagonists are evaluated with respect to their ability to facilitate wound healing according to procedures known in the art. If desired, zpep14 polypeptide performance in this regard can be compared to growth factors, such as EGF, NGF, TGF-α, TGF-β, insulin, IGF-I, IGF-II, fibroblast growth factor (FGF) and the like. Moreover, the effects of zpep14 polypeptides, agonists or antagonists thereof can be evaluated with respect to their ability to enhance wound contractility involved in wound healing. In addition, zpep14 polypeptides or agonists or antagonists thereof may be evaluated in combination with one or more growth factors to identify synergistic effects.

The molecules of the present invention are useful as components of defined cell culture media, as described herein, and may be used alone or in combination with other cytokines and hormones to replace serum that is commonly used in cell culture. Molecules of the present invention are particularly useful in specifically promoting the growth, development, differentiation, and/or maturation of ovarian cells in culture, and may also prove useful in the study of the ovarian cycle, reproductive function, ovarian and testicular cell-cell interactions, sperm capacitation and fertilization.

In addition, the present invention also provides methods for studying steroidogenesis and steroid hormone secretion. Such methods generally comprise incubating ovarian cells in culture medium comprising zpep14 polypeptides, monoclonal antibodies, agonists or antagonists thereof with and without gonadotropins and/or steroid hormones, and subsequently observing protein and steroid secretion. Exemplary gonadotropin hormones include luteinizing hormone and follicle stimulating hormone (Rouillier et al., Mol. Reprod. Dev. 50:170-7, 1998). Exemplary steroid hormones include estradiol, androstenedione, and progesterone. Effects of zpep14 on steroidogenesis or steroid secretion can be determined by methods known in the art, such as radioimmunoassay (to detect levels of estradiol, androstenedione, progesterone, and the like), and immunoradiometric assay (IRMA).

Molecules expressed in the uterus, testis and prostate, such as zpep14 polypeptide, and which may modulate hormones, hormone receptors, growth factors, or cell-cell interactions, of the reproductive cascade or are involved in oocyte or ovarian development, spermatogenesis, or the like, would be useful as markers for cancer of reproductive organs and as therapeutic agents for hormone-dependent cancers, by inhibiting hormone-dependent growth and/or development of tumor cells. Human reproductive system cancers such as ovarian, uterine, cervical, testicular and prostate cancers are common. Moreover, receptors for steroid hormones involved in the reproductive cascade are found in human tumors and tumor cell lines (breast, prostate, endometrial, ovarian, kidney, and pancreatic tumors) (Kakar et al., Mol. Cell. Endocrinol., 106:145-49, 1994; Kakar and Jennes, Cancer Letts., 98:57-62, 1995). Thus, expression of zpep14 in reproductive tissues suggests that polypeptides of the present invention would be useful in diagnostic methods for the detection and monitoring of reproductive cancers.

Diagnostic methods of the present invention involve the detection of zpep14 polypeptides in the serum or tissue biopsy of a patient undergoing analysis of reproductive function or evaluation for possible reproductive cancers, e.g., uterine, testicular or prostate cancer. Such polypeptides can be detected using immunoassay techniques and antibodies, described herein, that are capable of recognizing zpep14 polypeptide epitopes. More specifically, the present invention contemplates methods for detecting zpep14 polypeptides comprising:

exposing a test sample potentially containing zpep14 polypeptides to an antibody attached to a solid support, wherein said antibody binds to a first epitope of a zpep14 polypeptide;

washing the immobilized antibody-polypeptide to remove unbound contaminants;

exposing the immobilized antibody-polypeptide to a second antibody directed to a second epitope of a zpep14 polypeptide, wherein the second antibody is associated with a detectable label; and

detecting the detectable label. Altered levels of zpep14 polypeptides in a test sample, such as serum sweat, saliva, biopsy, and the like, can be monitored as an indication of reproductive function or of reproductive cancer or disease, when compared against a normal control.

Additional methods using probes or primers derived, for example, from the nucleotide sequences disclosed herein can also be used to detect zpep14 expression in a patient sample, such as a blood, saliva, sweat, biopsy, tissue sample, or the like. For example, probes can be hybridized to tumor tissues and the hybridized complex detected by in situ hybridization. Zpep14 sequences can also be detected by PCR amplification using cDNA generated by reverse translation of sample mRNA as a template (PCR Primer A Laboratory Manual, Dieffenbach and Dveksler, eds., Cold Spring Harbor Press, 1995). When compared with a normal control, both increases or decreases of zpep14 expression in a patient sample, relative to that of a control, can be monitored and used as an indicator or diagnostic for disease.

Moreover, the activity and effect of zpep14 polypeptides on tumor progression and metastasis can be measured in vivo. Several syngeneic mouse models have been developed to study the influence of polypeptides, compounds or other treatments on tumor progression. In these models, tumor cells passaged in culture are implanted into mice of the same strain as the tumor donor. The cells will develop into tumors having similar characteristics in the recipient mice, and metastasis will also occur in some of the models. Appropriate tumor models for our studies include the Lewis lung carcinoma (ATCC No. CRL-1642) and B16 melanoma (ATCC No. CRL-6323), amongst others. These are both commonly used tumor lines, syngeneic to the C57BL6 mouse, that are readily cultured and manipulated in vitro. Tumors resulting from implantation of either of these cell lines are capable of metastasis to the lung in C57BL6 mice. The Lewis lung carcinoma model has recently been used in mice to identify an inhibitor of angiogenesis (O'Reilly M S, et al. Cell 79: 315-328,1994). C57BL6/J mice are treated with an experimental agent either through daily injection of recombinant protein, agonist or antagonist or a one time injection of recombinant adenovirus. Three days following this treatment, 10⁵ to 10⁶ cells are implanted under the dorsal skin. Alternatively, the cells themselves may be infected with recombinant adenovirus, such as one expressing zpep14, before implantation so that the protein is synthesized at the tumor site or intracellularly, rather than systemically. The mice normally develop visible tumors within 5 days. The tumors are allowed to grow for a period of up to 3 weeks, during which time they may reach a size of 1500-1800 mm³ in the control treated group. Tumor size and body weight are carefully monitored throughout the experiment. At the time of sacrifice, the tumor is removed and weighed along with the lungs and the liver. The lung weight has been shown to correlate well with metastatic tumor burden. As an additional measure, lung surface metastases are counted. The resected tumor, lungs and liver are prepared for histopathological examination, immunohistochemistry, and in situ hybridization, using methods known in the art and described herein. The influence of the expressed polypeptide in question, e.g., zpep14, on the ability of the tumor to recruit vasculature and undergo metastasis can thus be assessed. In addition, aside from using adenovirus, the implanted cells can be transiently transfected with zpep14. Use of stable zpep14 transfectants as well as use of induceable promoters to activate zpep14 expression in vivo are known in the art and can be used in this system to assess zpep14 induction of metastasis. Moreover, purified zpep14, synthesized zpep14 peptides, or zpep14-conditioned media can be directly injected in to this mouse model, and hence be used in this system. For general reference see, O'Reilly M S, et al. Cell 79:315-328, 1994; and Rusciano D, et al. Murine Models of Liver Metastasis. Invasion Metastasis 14:349-361, 1995.

Polynucleotides encoding zpep14 polypeptides are useful within gene therapy or gene transfer applications where it is desired to increase or inhibit zpep14 activity. If a mammal has a mutated or absent zpep14 gene, the zpep14 gene can be introduced into the cells of the mammal. In one embodiment, a gene encoding a zpep14 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a zpep14 gene can be introduced in a retroviral vector, e.g., as described in Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; International Patent Publication No. WO 95/07358, published Mar. 16, 1995 by Dougherty et al.; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

It is possible to remove the target cells from the body; to introduce the vector as a naked DNA plasmid; and then to re-implant the transformed cells into the body. Naked DNA vectors for gene therapy or gene transfer can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Antisense methodology can be used to inhibit zpep14 gene transcription, such as to inhibit cell proliferation in vivo. Polynucleotides that are complementary to a segment of a zpep14-encoding polynucleotide (e.g., a polynucleotide as set froth in SEQ ID NO:1) are designed to bind to zpep14-encoding mRNA and to inhibit translation of such mRNA. Such antisense polynucleotides are used to inhibit expression of zpep14 polypeptide-encoding genes in cell culture or in a subject.

The present invention also provides reagents which will find use in diagnostic applications. For example, the zpep14 gene, a probe comprising zpep14 DNA or RNA or a subsequence thereof can be used to determine if the zpep14 gene is present on chromosome 7 or if a mutation has occurred. Zpep14 is located at the 7p12 region of chromosome 7 (See, Example 3). Detectable chromosomal aberrations at the zpep14 gene locus include, but are not limited to, aneuploidy, gene copy number changes, insertions, deletions, restriction site changes and rearrangements. Such aberrations can be detected using polynucleotides of the present invention by employing molecular genetic techniques, such as restriction fragment length polymorphism (RFLP) analysis, fluorescence in situ hybridization methods, short tandem repeat (STR) analysis employing PCR techniques, and other genetic linkage analysis techniques known in the art (Sambrook et al., ibid; Ausubel et. al., ibi.; Marian, Chest 108:255-65, 1995).

The precise knowledge of a gene's position can be useful for a number of purposes, including: 1) determining if a sequence is part of an existing contig and obtaining additional surrounding genetic sequences in various forms, such as YACs, BACs or cDNA clones; 2) providing a possible candidate gene for an inheritable disease which shows linkage to the same chromosomal region; and 3) cross-referencing model organisms, such as mouse, which may aid in determining what function a particular gene might have.

The zpep14 gene is located at the 7p12 region of chromosome 7. Several genes of known function or correlated with human disease map to this region. For example, the glioblastoma amplified sequence (GBAS) that maps to the 7p12 region is amplified in glioblastomas, a common spinal and brain malignancy (Wang, X. et al., Genomics 49:448-451, 1998). Thus, zpep14 polynucleotide probes can be used to detect abnormalities or genotypes associated with glioblastoma. Further, zpep14 polynucleotide probes can be used to detect abnormalities or genotypes associated with a dominant form of non-insulin dependent diabetes mellitus (NIDDM) called maturity onset diabetes of the young (MODY) and hyperinsulinism, where a susceptibility marker maps to the glucokinase gene at 7p15-p13 (Froguel, P. et al, Nature 356:162-164, 1992). In addition, zpep14 polynucleotide probes can be used to detect abnormalities or genotypes associated with hand-foot-uterus syndrome, where a susceptibility marker maps to 7p15-7p14.2 (Stem A. M. et al, J. Pediat. 77:109-116, 1970; Mortlock, D. P. and Innis, J. W., Nature Genet. 15:179-181, 1997). Moreover, amongst other genetic loci, those for Wilms tumor suppressor (7p15-p11.2), Charcot-Marie-Tooth disease, neuronal type D (7p14), invasion and metastasis factors (7p12-cen), and myoppathy due to phosphoglycerate mutase deficiency (7p13-p12.3), all manifest themselves in human disease states as well as map to this region of the human genome. See the Online Mendellian Inheritance of Man (OMIM) gene map, and references therein, for this region of chromosome 7 on a publicly available WWW server (http://www3.ncbi.nlm.nih.gov/htbin-post/Omim/getmap?chromosome=7p12). All of these serve as possible candidate genes for an inheritable disease which show linkage to the same chromosomal region as the zpep14 gene.

Similarly, defects in the zpep14 locus itself may result in a heritable human disease state. Molecules of the present invention, such as the polypeptides, antagonists, agonists, polynucleotides and antibodies of the present invention would aid in the detection, diagnosis prevention, and treatment associated with a zpep14 genetic defect.

Mice engineered to express the zpep14 gene, referred to as “transgenic mice,” and mice that exhibit a complete absence of zpep14 gene function, referred to as “knockout mice,” may also be generated (Snouwaert et al., Science 257:1083, 1992; Lowell et al., Nature 366:740-42, 1993; Capecchi, M. R., Science 244: 1288-1292, 1989; Palmiter, R. D. et al. Annu Rev Genet. 20: 465-499, 1986). For example, transgenic mice that over-express zpep14, either ubiquitously or under a tissue-specific or tissue-restricted promoter can be used to ask whether over-expression causes a phenotype. For example, over-expression of a wild-type zpep14 polypeptide, polypeptide fragment or a mutant thereof may alter normal cellular processes, resulting in a phenotype that identifies a tissue in which zpep14 expression is functionally relevant and may indicate a therapeutic target for the zpep14, its agonists or antagonists. For example, a preferred transgenic mouse to engineer is one that over-expresses the zpep14 mature polypeptide (residue 17 (Arg) to residue 188 (Asn) of SEQ ID NO:2). Transgenic mice engineered to over-expresses zpep14 polypeptides-1 thorough -9 can also be used. Moreover, such over-expression may result in a phenotype that shows similarity with human diseases. Similarly, knockout zpep14 mice can be used to determine where zpep14 is absolutely required in vivo. The phenotype of knockout mice is predictive of the in vivo effects of that a zpep14 antagonist, such as those described herein, may have. The human zpep14 cDNA can be used to isolate murine zpep14 mRNA, cDNA and genomic DNA, which are subsequently used to generate knockout mice. The mouse zpep14 sequences used to generate knockout mice, sucha se the mouse zpep14 mature polypeptide (residue 17 (Gly) to residue 187 (Asn) of SEQ ID NO:2) are described herein. Transgenic mice engineered to over-expresses mouse zpep14 polypeptides-1m, -5m, -6m, -7m, -9m, or mouse polypeptides corresponding to the human polypeptides-1 through -9 can also be used. These mice may be employed to study the zpep14 gene and the protein encoded thereby in an in vivo system, and can be used as in vivo models for corresponding human diseases. Moreover, transgenic mice expression of zpep14 antisense polynucleotides or ribozymes directed against zpep14, described herein, can be used analogously to transgenic mice described above.

For pharmaceutical use, the proteins of the present invention are formulated for parenteral, particularly intravenous or subcutaneous, delivery according to conventional methods. Intravenous administration will be by bolus injection or infusion over a typical period of one to several hours. In general, pharmaceutical formulations will include a zpep14 polypeptide in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Therapeutic doses will generally be in the range of 0.1 to 100 μg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. The proteins may be administered for acute treatment, over one week or less, often over a period of one to three days or may be used in chronic treatment, over several months or years.

The invention is further illustrated by the following non-limiting examples.

### EXAMPLES

### Example 1

Identification of zpep14 Using an EST Sequence to Obtain Full-length zpep14

Scanning of translated DNA databases resulted in identification of an expressed sequence tag (EST) sequence. The initial EST sequence was contained in a plasmid, and contained a partial 3′ sequence. 5′RACE was carried out with primers ZC18,791 (SEQ ID NO:4) and ZC18,792 (SEQ ID NO:5) using lymph node cDNA prepared from lymph node RNA (Clontech) using a Marathon cDNA kit (Clontech). PCR conditions were as follows: one cycle at 94° C. for 1.5 min.; 32 cycles at 94° C. for 15 sec., and 68° C. for one cycle at 72° C. for 10 min.; followed by a 4° C. hold. The PCR reaction was electrophoresed on a 1.5% agarose gel and a 180 bp band was excised and gel purified using QiaexII reagents (Qiagen) according to the manufacturer's protocol. The excised 180 bp band was directly ligated into a TA vector (Invitrogen).

Using primers to the original EST and the 5′ RACE product, a single clone containing the complete full length zpep14 sequence was isolated. Primers ZC18,791 (SEQ ID NO:4) and ZC20,516 (SEQ ID NO:6) using lymph node cDNA described above were used in a PCR reaction under the following conditions: one cycle at 94° C. for 1.5 min.; 35 cycles at 94° C. for 15 sec., and 62° C. for 20 seconds, and 72° C. for 1 minute; one cycle at 72° C. for 10 min.; followed by a 4° C. hold. The PCR reaction was electrophoresed on a 1.0% agarose gel and an approximately 500 bp band was excised and gel purified using QiaexII reagents (Qiagen) according to the manufacturer's protocol. A portion of the excised approximately 500 bp band was directly ligated into a TA vector (Invitrogen) at 16° C. overnight. A portion of the ligation reaction was electroporated into E. coli DH10B cells (Gibco/BRL). Miniprep DNA from transformant clones were screened for insert and a single clone containing the complete full length zpep14 sequence of approximately 580 bp was isolated.

Sequence analysis was performed, confirming the EST sequence of the cDNA from which the EST originated, the 5′ extension of the initial EST sequence, as well as the final zpep14 full-length cDNA. The following primers were used for the sequence analysis: ZC694 (SEQ ID NO:7), ZC6,768 (SEQ ID NO:8), ZC7,710 (SEQ ID NO:9), ZC3424 (SEQ ID NO:10).

### Example 2

Tissue Distribution

Northern blot analysis was performed using Human Multiple Tissue Northern™ Blots (MTN I, MTN II, and MTN III) (Clontech). Heart and fetal brain cDNA was prepared from heart and fetal brain RNA (Clontech) using a Marathon cDNA kit (Clontech). This heart and fetal brain cDNA was used in a PCR reaction with oligos ZC18,143 (SEQ ID NO:11) and ZC18,144 (SEQ ID NO:12) as primers. PCR conditions were as follows: 94° C. for 1.5 minutes; 35 cycles at 94° C. for 15 seconds then 60° C. for seconds; 72° C. for 10 minutes; 4° C. overnight A sample of the PCR reaction product was run on a 4% agarose gel. A band of the expected size of 215 bp was seen. The 215 bp PCR fragment, was gel purified using a commercially available kit (QiaexII™; Qiagen) and then radioactively labeled with ³²P-dCTP using Rediprime II™ (Amersham), a random prime labeling system, according to the manufacturer's specifications. The probe was then purified using a Nuc-Trap™ column (Stratagene) according to the manufacturer's instructions. ExpressHyb™ (Clontech) solution was used for prehybridization and as a hybridizing solution for the Northern blots. Hybridization took place overnight at 65° C. using 1-2×10⁶ cpm/ml of labeled probe. The blots were then washed 4 times for 15 minutes in 2×SSC/1% SDS at 25° C., followed by a wash in 0.1×SSC/0.1% SDS at 50° C. for one hour, and then twice more in 0.1×SSC/0.1% SDS at 50° C. for 30 minutes each. A transcript of approximately 1 kb was detected at high levels in prostate, testis and uterus, moderate levels in heart, thyroid, spleen, colon and pancreas, and low levels in other tissues.

Dot Blots were also performed using Human RNA Master Blots™ (Clontech). The methods and conditions for the Dot Blots are the same as for the Multiple Tissue Blots described above. Dot blot had strong signals in prostate, stomach and liver, and moderate signals in heart and lower in other tissues.

### Example 3

PCR-Based Chromosomal Mapping of the zpep14 Gene

Zpep14 was mapped to chromosome 7 using the commercially available version of the “Stanford G3 Radiation Hybrid Mapping Panel” (Research Genetics, Inc., Huntsville, Ala.). The “Stanford G3 RH Panel” contains PCRable DNAs from each of 83 radiation hybrid clones of the whole human genome, plus two control DNAs (the RM donor and the A3 recipient). A publicly available WWW server (http://shgc-www.stanford.edu) allows chromosomal localization of markers.

For the mapping of Zpep14 with the “Stanford G3 RH Panel”, 20 μl reactions were set up in a 96-well microtiter plate (Stratagene, La Jolla, Calif.) and used in a “RoboCycler Gradient 96” thermal cycler (Stratagene). Each of the 85 PCR reactions consisted of 2 μl 10×KlenTaq PCR reaction buffer (Clontech Laboratories, Inc., Palo Alto, Calif.), 1.6 μl dNTPs mix (2.5 mM each, PERKIN-ELMER, Foster City, Calif.), 1 μl sense primer, ZC 21,355, (SEQ ID NO:13), 1 μl antisense primer, ZC 21,356, (SEQ ID NO:14), 2 μl “RediLoad” (Research Genetics, Inc., Huntsville, Ala.), 0.4 μl 50×Advantage KlenTaq Polymerase Mix (Clontech), 25 ng of DNA from an individual hybrid clone or control and ddH₂O for a total volume of 20 μl. The reactions were overlaid with an equal amount of mineral oil and sealed. The PCR cycler conditions were as follows: an initial 1 cycle 5 minute denaturation at 94 ° C., 35 cycles of a 45 seconds denaturation at 94° C., 45 seconds annealing at 62° C. and 1 minute and 15 seconds extension at 72° C., followed by a final 1 cycle extension of 7 minutes at 72° C. The reactions were separated by electrophoresis on a 2% agarose gel (Life Technologies, Gaithersburg, Md.).

The results showed linkage of zpep14 to the framework marker SHGC-33787 with a LOD score of >15 and at a distance of 4 cR_{—}10000 from the marker. The use of surrounding markers positions Zpep14 in the 7p12 region on the integrated LDB chromosome 7 map (The Genetic Location Database, University of Southhampton, WWW server: http://cedar.genetics. soton.ac.uk/public_html/).

### Example 4

Identification and Isolation of Murine zpep14 Using the Human zpep14 Sequence

Scanning of translated murine DNA databases using the human zpep14 sequence (SEQ ID NO:1) (Example 1) resulted in identification of several expressed sequence tag (EST) sequences. One of the murine ESTs were used as seed sequence to search for contigs. The most 5′end EST in the contig was then compared to the human zpep14 sequence to examine the full length status of this EST. The EST was then purchased After alignment indicating that the EST is potential full length mouse zpep14. Sequence analysis was performed, confirming the EST sequence of the murine cDNA from which the EST originated was a mouse zpep14 full-length cDNA. The following primers were used for the sequence analysis: ZC694 (SEQ ID NO:7), ZC6,768 (SEQ ID NO:8), ZC20,672 (SEQ ID NO:15), and ZC20,622 (SEQ ID NO:16). The murine zpep14 polynucleotide sequence is shown in SEQ ID NO:17, and the corresponding polypeptide sequence shown in SEQ ID NO:18.

### Example 5

Construct for Generating zpep14 Transgenic Mice

Oligonucleotides were designed to generate a PCR fragment containing a consensus Kozak sequence and the exact zpep14 coding region. These oligonucleotides were designed with an FseI site at the 5′ end and an AscI site at the 3′ end to facilitate cloning into pTG12-8, our standard transgenic vector. The pTG12-8 vector contains the mouse MT-1 1 promoter and a 5′ rat insulin II intron upstream of the FseI site.

PCR reactions were carried out using Advantage® cDNA polymerase (Clontech) to amplify a zpep14 cDNA fragment. About 200 ng human zpep14 polynucleotide template (Example 1), and oligonucleotides ZC20,886 (SEQ ID NO:19) and ZC20,887 (SEQ ID NO:20) were used in the PCR reaction. PCR reaction conditions were as follows: 95° C. for 5 minutes,; 15 cycles of 95° C. for 60 seconds, 61° C. for 60 seconds, and 72° C. for 90 seconds; and 72° C. for 7 minutes; followed by a 4° C. hold. PCR products were separated by agarose gel electrophoresis and purified using a QiaQuick™ (Qiagen) gel extraction kit. The isolated, approximately 567 bp, DNA fragment was digested with FseI and AscI (Boerhinger-Mannheim), ethanol precipitated and ligated into pTG12-8 that was previously digested with FseI and AscI. The pTG12-8 plasmid, designed for expression of a gene of interest in transgenic mice, contains an expression cassette flanked by 10 kb of MT-1 5′ DNA and 7 kb of MT-1 3′ DNA. The expression cassette comprises the MT-1 promoter, the rat insulin II intron, a polylinker for the insertion of the desired clone, and the human growth hormone poly A sequence.

About one microliter of the ligation reaction was electroporated into DH10B ElectroMax™ competent cells (GIBCO BRL, Gaithersburg, Md.) according to manufacturer's direction and plated onto LB plates containing 100 μg/ml ampicillin, and incubated overnight. Colonies were picked and grown in LB media containing 100 μg/ml ampicillin. Miniprep DNA was prepared from the picked clones and screened for the zpep14 insert by restriction digestion with EcoRI, and subsequent agarose gel electrophoresis. Maxipreps of the correct pTG-zpep14 construct, as verified by sequence analysis, were performed. A SalI fragment containing with 5′ and 3′ flanking sequences, the MT-1 promoter, the rat insulin II intron, zpep14 cDNA and the human growth hormone poly A sequence was prepared to be used for microinjection into fertilized murine oocytes.

### Example 6

Chemical Synthesis and Purification of Human Zpep14 Peptides: zpep14 Polypeptide-3 and Polypeptide-7

Zpep14 polypeptide-3 (Zpep14-3) and Zpep14 polypeptide-7 (Zpep14-7) were synthesized by solid phase peptide synthesis using the ABI/PE Peptide Synthesizer model 431A (Applied Biosytems/Perkin Elmer (ABI/PE, Foster City, Calif.). The Zpep14-3 peptide sequence is shown in SEQ ID NO:21 and corresponds to amino acid residues 140 (Gln) to amino acid residue 171 (Gly) of SEQ ID NO:2. The Zpeptide14-7 sequence is shown in SEQ ID NO:22 and corresponds to amino acid residues 174 (Ile) to amino acid residue 188 (Asn) of SEQ ID NO:2.

Fmoc-Amide resin was used for synthesis of the Zpep14-3 peptide and Fmoc-Asparagine resin was used for the Zpep14-7 peptide. The Fmoc-Amide resin (0.68 mmol/g) and the Fmoc-Asparagine resin (0.75 mmol/g) were purchased from ABI/PE. The amino acids were purchased from AnaSpec, Inc., San Jose, Calif. in pre-weighed, 1 mmol cartridges. All the reagents except piperidine were purchased from ABI/PE. The piperidine was purchased from Aldrich, St. Louis Mo. Synthesis procedure was taken from the ABI Model 431A manual. Double coupling cycles were used during the high aggregation portion of the sequence, as predicted by Peptide Companion software (Peptides International, Louisville, Ky.).

The peptides were cleaved from the solid phase following the standard TFA cleavage procedure as outlined in the Peptide Cleavage protocol manual published by ABI/PE. Purification of the peptides were by RP-HPLC using a C18, 10 mm preparative column. Eluted fractions from the column were collected and analyzed for correct mass and purity by electrospray mass spectrometry. The analysis results indicated that the Zpep14-3 and Zpep14-7 peptides were present and pure in one of the pools from the HPLC purification step. The pools containing each of the peptides were retained and lyophilized.

Post lyophilization, the Zpep14-3 and the Zpep14-7 peptides were analyzed for purity using analytical HPLC. The analytical HPLC column used was a Vydac 10 cm, 5 um column. The analysis resulted in 95% purity for both Zpep14-3 and Zpep14-7 peptides. These peptides were prepared for use in subsequent biological assays.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polynucleotide encoding a zpep14 polypeptide comprising a sequence of amino acid residues that is at least 90% identical to an amino acid sequence selected from the group consisting of:
(a) the amino acid sequence as shown in SEQ ID NO:2 from amino acid number 17 (Arg) to amino acid number 92 (Cys);
(b) the amino acid sequence as shown in SEQ ID NO:2 from amino acid number 96 (Asn) to amino acid number 137 (Ile);
(c) the amino acid sequence as shown in SEQ ID NO:2 from amino acid number 140 (Gln) to amino acid number 171 (Gly);
(d) the amino acid sequence as shown in SEQ ID NO:2 from amino acid number 17 (Arg) to amino acid number 137 (Ile);
(e) the amino acid sequence as shown in SEQ ID NO:2 from amino acid number 17 (Arg) to amino acid number 171 (Gly);
(f) the amino acid sequence as shown in SEQ ID NO:2 from amino acid number 96 (Asn) to amino acid number 171 (Gly);
(g) the amino acid sequence as shown in SEQ ID NO:2 from amino acid number 174 (Ile) to amino acid number 188 (Asn);
(h) the amino acid sequence as shown in SEQ ID NO:2 from amino acid number 140 (Gln) to amino acid number 188 (Asn);
(i) the amino acid sequence as shown in SEQ ID NO:2 from amino acid number 96 (Asn) to amino acid number 188 (Asn);
(j) the amino acid sequence as shown in SEQ ID NO:2 from amino acid number 17 (Arg) to amino acid number 188 (Asn); and
(k) the amino acid sequence as shown in SEQ ID NO:2 from amino acid number 1 (Met) to amino acid number 188 (Asn),
wherein the amino acid percent identity is determined using a FASTA program with ktup=1, gap opening penalty=10, gap extension penalty=1, and substitution matrix=BLOSUM62, with other parameters set as default.

2. An isolated polynucleotide according to claim 1, wherein the polynucleotide is selected from the group consisting of:
(a) a polynucleotide sequence as shown in SEQ ID NO:1 from nucleotide 53 to nucleotide 280;
(b) a polynucleotide sequence as shown in SEQ ID NO:1 from nucleotide 290 to nucleotide 415;
(c) a polynucleotide sequence as shown in SEQ ID NO:1 from nucleotide 422 to nucleotide 517;
(d) a polynucleotide sequence as shown in SEQ ID NO:1 from nucleotide 53 to nucleotide 415;
(e) a polynucleotide sequence as shown in SEQ ID NO:1 from nucleotide 53 to nucleotide 517;
(f) a polynucleotide sequence as shown in SEQ ID NO:1 from nucleotide 290 to nucleotide 517;
(g) a polynucleotide sequence as shown in SEQ ID NO:1 from nucleotide 524 to nucleotide 568;
(h) a polynucleotide sequence as shown in SEQ ID NO:1 from nucleotide 422 to nucleotide 568;
(i) a polynucleotide sequence as shown in SEQ ID NO:1 from nucleotide 290 to nucleotide 568;
(j) a polynucleotide sequence as shown in SEQ ID NO:1 from nucleotide 53 to nucleotide 568;
(k) a polynucleotide sequence as shown in SEQ ID NO:1 from nucleotide 5 to nucleotide 568; and
(l) a polynucleotide sequence complementary to (a) through (k).

3. An isolated polynucleotide sequence according to claim 1, wherein the polynucleotide comprises nucleotide 1 to nucleotide 564 of SEQ ID NO:3.

4. An isolated polynucleotide according to claim 1, wherein the zpep14 polypeptide comprises a sequence of amino acid residues selected from the group consisting of:
(a) the amino acid sequence as shown in SEQ ID NO:2 from amino acid number 17 (Arg) to amino acid number 92 (Cys);
(b) the amino acid sequence as shown in SEQ ID NO:2 from amino acid number 96 (Asn) to amino acid number 137 (Ile);
(c) the amino acid sequence as shown in SEQ ID NO:2 from amino acid number 140 (Gln) to amino acid number 171 (Gly);
(d) the amino acid sequence as shown in SEQ ID NO:2 from amino acid number 17 (Arg) to amino acid number 137 (Ile);
(e) the amino acid sequence as shown in SEQ ID NO:2 from amino acid number 17 (Arg) to amino acid number 171 (Gly);
(f) the amino acid sequence as shown in SEQ ID NO:2 from amino acid number 96 (Asn) to amino acid number 171 (Gly);
(g) the amino acid sequence as shown in SEQ ID NO:2 from amino acid number 174 (Ile) to amino acid number 188 (Asn);
(h) the amino acid sequence as shown in SEQ ID NO:2 from amino acid number 140 (Gln) to amino acid number 188 (Asn);
(i) the amino acid sequence as shown in SEQ ID NO:2 from amino acid number 96 (Asn) to amino acid number 188 (Asn);
(j) the amino acid sequence as shown in SEQ ID NO:2 from amino acid number 17 (Arg) to amino acid number 188 (Asn); and
(k) the amino acid sequence as shown in SEQ ID NO:2 from amino acid number 1 (Met) to amino acid number 188 (Asn).

5. An isolated polynucleotide according to claim 4, wherein the zpep14 polypeptide consists of a sequence of amino acid residues as shown in SEQ ID NO:2 from amino acid number 96 (Asn) to amino acid number 137 (lIe).

6. An expression vector comprising the following operably linked elements:
a transcription promoter;
a DNA segment encoding a zpep14 polypeptide that is at least 90% identical to an amino acid sequence as shown in SEQ ID NO:2 from amino acid number 17 (Arg) to amino acid number 188 (Asn); and
a transcription terminator.

7. An expression vector according to claim 6, further comprising a secretory signal sequence operably linked to the DNA segment.

8. A cultured cell into which has been introduced an expression vector according to claim 6, wherein the cell expresses a polypeptide encoded by the DNA segment.

9. A DNA construct encoding a fusion protein, the DNA construct comprising:
a first DNA segment encoding a polypeptide that is at least 90% identical to a sequence of amino acid residues selected from the group consisting of:
(a) the amino acid sequence of SEQ ID NO:2 from residue number 1 (Met) to residue number 16 (Ala));
(b) the amino acid sequence as shown in SEQ ID NO:2 from amino acid number 17 (Arg) to amino acid number 92 (Cys);
(c) the amino acid sequence as shown in SEQ ID NO:2 from amino acid number 96 (Asn) to amino acid number 137 (Ile);
(d) the amino acid sequence as shown in SEQ ID NO:2 from amino acid number 140 (Gln) to amino acid number 171 (Gly);
(e) the amino acid sequence as shown in SEQ ID NO:2 from amino acid number 17 (Arg) to amino acid number 137 (Ile);
(f) the amino acid sequence as shown in SEQ ID NO:2 from amino acid number 17 (Arg) to amino acid number 171 (Gly);
(g) the amino acid sequence as shown in SEQ ID NO:2 from amino acid number 96 (Asn) to amino acid number 171 (Gly);
(h) the amino acid sequence as shown in SEQ ID NO:2 from amino acid number 174 (Ile) to amino acid number 188 (Asn);
(i) the amino acid sequence as shown in SEQ ID NO:2 from amino acid number 140 (Gln) to amino acid number 188 (Asn);
(j) the amino acid sequence as shown in SEQ ID NO:2 from amino acid number 96 (Asn) to amino acid number 188 (Asn);
(k) the amino acid sequence as shown in SEQ ID NO:2 from amino acid number 17 (Arg) to amino acid number 188 (Asn); and
(l) the amino acid sequence as shown in SEQ ID NO:2 from amino acid number 1 (Met) to amino acid number 188 (Asn). at least one other DNA segment encoding an additional polypeptide,
wherein the first and other DNA segments are connected in-frame; and
encode the fusion protein.

10. A fusion protein produced by a method comprising:
culturing a host cell into which has been introduced a vector comprising the following operably linked elements:
(a) a transcriptional promoter;
(b) a DNA construct encoding a fusion protein according to claim 9; and
(c) a transcriptional terminator; and
recovering the protein encoded by the DNA segment.

11. An isolated polypeptide comprising a sequence of amino acid residues that is at least 90% identical to an amino acid sequence selected from the group consisting of:
(a) the amino acid sequence as shown in SEQ ID NO:2 from amino acid number 17 (Arg) to amino acid number 92 (Cys);
(b) the amino acid sequence as shown in SEQ ID NO:2 from amino acid number 96 (Asn) to amino acid number 137 (Ile);
(c) the amino acid sequence as shown in SEQ ID NO:2 from amino acid number 140 (Gln) to amino acid number 171 (Gly);
(d) the amino acid sequence as shown in SEQ ID NO:2 from amino acid number 17 (Arg) to amino acid number 137 (Ile);
(e) the amino acid sequence as shown in SEQ ID NO:2 from amino acid number 17 (Arg) to amino acid number 171 (Gly);
(f) the amino acid sequence as shown in SEQ ID NO:2 from amino acid number 96 (Asn) to amino acid number 171 (Gly);
(g) the amino acid sequence as shown in SEQ ID NO:2 from amino acid number 174 (Ile) to amino acid number 188 (Asn);
(h) the amino acid sequence as shown in SEQ ID NO:2 from amino acid number 140 (Gln) to amino acid number 188 (Asn);
(i) the amino acid sequence as shown in SEQ ID NO:2 from amino acid number 96 (Asn) to amino acid number 188 (Asn);
(j) the amino acid sequence as shown in SEQ ID NO:2 from amino acid number 17 (Arg) to amino acid number 188 (Asn); and
(k) the amino acid sequence as shown in SEQ ID NO:2 from amino acid number 1 (Met) to amino acid number 188 (Asn),
wherein the amino acid percent identity is determined using a FASTA program with ktup=1, gap opening penalty=10, gap extension penalty=1, and substitution matrix=BLOSUM62, with other parameters set as default.

12. An isolated polypeptide according to claim 11, wherein the polypeptide comprising a sequence of amino acid residues that is selected from the group consisting of:
(a) the amino acid sequence as shown in SEQ ID NO:2 from amino acid number 17 (Arg) to amino acid number 92 (Cys);
(b) the amino acid sequence as shown in SEQ ID NO:2 from amino acid number 96 (Asn) to amino acid number 137 (Ile);
(c) the amino acid sequence as shown in SEQ ID NO:2 from amino acid number 140 (Gln) to amino acid number 171 (Gly);
(d) the amino acid sequence as shown in SEQ ID NO:2 from amino acid number 17 (Arg) to amino acid number 137 (Ile);
(e) the amino acid sequence as shown in SEQ ID NO:2 from amino acid number 17 (Arg) to amino acid number 171 (Gly);
(f) the amino acid sequence as shown in SEQ ID NO:2 from amino acid number 96 (Asn) to amino acid number 171 (Gly);
(g) the amino acid sequence as shown in SEQ ID NO:2 from amino acid number 174 (Ile) to amino acid number 188 (Asn);
(h) the amino acid sequence as shown in SEQ ID NO:2 from amino acid number 140 (Gln) to amino acid number 188 (Asn);
(i) the amino acid sequence as shown in SEQ ID NO:2 from amino acid number 96 (Asn) to amino acid number 188 (Asn);
(j) the amino acid sequence as shown in SEQ ID NO:2 from amino acid number 17 (Arg) to amino acid number 188 (Asn); and
(k) the amino acid sequence as shown in SEQ ID NO:2 from amino acid number 1 (Met) to amino acid number 188 (Asn).

13. An isolated polypeptide according to claim 12, wherein the sequence of amino acid residues is as shown in SEQ ID NO:2 from amino acid number 96 (Asn) to amino acid number 137 (Ile).

14. A method of producing a zpep14 polypeptide comprising:
culturing a cell according to claim 8; and
isolating the zpep14 polypeptide produced by the cell.

15. A method of detecting, in a test sample, the presence of a modulator of zpep14 protein activity, comprising:
transfecting a zpep14-responsive cell, with a reporter gene construct that is responsive to a zpep14-stimulated cellular pathway; and
producing a zpep14 polypeptide by the method of claim 14; and
adding the zpep14 polypeptide to the cell, in the presence and absence of a test sample; and
comparing levels of response to the zpep14 polypeptide, in the presence and absence of the test sample, by a biological or biochemical assay; and
determining from the comparison, the presence of the modulator of zpep14 activity in the test sample.

16. A method of producing an antibody to zpep14 polypeptide comprising the following steps in order:
inoculating an animal with a polypeptide selected from the group consisting of:
(a) a polypeptide consisting of 9 to 172 amino acids, wherein the polypeptide is at least 90% identical to a contiguous sequence of amino acids in SEQ ID NO:2 from amino acid number 17 (Arg) to amino acid number 188 (Asn);
(b) a polypeptide consisting of the amino acid sequence of SEQ ID NO:2 from amino acid number 17 (Arg) to amino acid number 188 (Asn);
(c) a polypeptide according to claim 11;
(d) a polypeptide consisting of amino acid number 90 (Asn) to amino acid number 95 (Arg) of SEQ ID NO:2;
(e) a polypeptide consisting amino acid number 128 (Glu) to amino acid number 133 (Glu) of SEQ ID NO:2;
(f) a polypeptide consisting of amino acid number 167 (Glu) to amino acid number 172 (Lys) of SEQ ID NO:2;
(g) a polypeptide consisting of amino acid number 175 (Ile) to amino acid number 180 (Lys) of SEQ ID NO:2; and
(h) a polypeptide consisting of amino acid number 176 (Glu) to amino acid number 181 (Arg) of SEQ ID NO:2; and
and wherein the polypeptide elicits an immune response in the animal to produce the antibody; and isolating the antibody from the animal.

17. An antibody produced by the method of claim 16, which binds to a zpep14 polypeptide.

18. The antibody of claim 17, wherein the antibody is a monoclonal antibody.

19. An antibody which binds to a polypeptide of claim 11.

20. An isolated polynucleotide encoding a zpep14 polypeptide comprising a sequence of amino acid residues that is at least 90% identical to an amino acid sequence selected from the group consisting of:
(a) the amino acid sequence as shown in SEQ ID NO:18 from amino acid number 17 (Gly) to amino acid number 91 (Cys);
(b) the amino acid sequence as shown in SEQ ID NO:18 from amino acid number 17 (Gly) to amino acid number 170 (Gly);
(c) the amino acid sequence as shown in SEQ ID NO:18 from amino acid number 95 (Asn) to amino acid number 170 (Gly);
(d) the amino acid sequence as shown in SEQ ID NO:18 from amino acid number 173 (Ile) to amino acid number 187 (Asn);
(e) the amino acid sequence as shown in SEQ ID NO:18 from amino acid number 17 (Gly) to amino acid number 187 (Asn); and
(f) the amino acid sequence as shown in SEQ ID NO:18 from amino acid number 1 (Met) to amino acid number 187 (Asn),
wherein the amino acid percent identity is determined using a FASTA program with ktup=1, gap opening penalty=10, gap extension penalty=1, and substitution matrix=BLOSUM62, with other parameters set as default.

21. An isolated polynucleotide according to claim 20, wherein the polynucleotide is selected from the group consisting of:
(a) a polynucleotide sequence as shown in SEQ ID NO:17 from nucleotide 96 to nucleotide 320;
(b) a polynucleotide sequence as shown in SEQ ID NO:17 from nucleotide 96 to nucleotide 557;
(c) a polynucleotide sequence as shown in SEQ ID NO:17 from nucleotide 330 to nucleotide 557;
(d) a polynucleotide sequence as shown in SEQ ID NO:17 from nucleotide 564 to nucleotide 608;
(e) a polynucleotide sequence as shown in SEQ ID NO:17 from nucleotide 96 to nucleotide 608;
(f) a polynucleotide sequence as shown in SEQ ID NO:17 from nucleotide 48 to nucleotide 608; and
(g) a polynucleotide sequence complementary to (a) through (k).

22. An isolated polynucleotide according to claim 20, wherein the zpep14 polypeptide comprises a sequence of amino acid residues selected from the group consisting of:
(a) the amino acid sequence as shown in SEQ ID NO:18 from amino acid number 17 (Gly) to amino acid number 91 (Cys);
(b) the amino acid sequence as shown in SEQ ID NO:18 from amino acid number 17 (Gly) to amino acid number 170 (Gly);
(c) the amino acid sequence as shown in SEQ ID NO:18 from amino acid number 95 (Asn) to amino acid number 170 (Gly);
(d) the amino acid sequence as shown in SEQ ID NO:18 from amino acid number 173 (Ile) to amino acid number 187 (Asn);
(e) the amino acid sequence as shown in SEQ ID NO:18 from amino acid number 17 (Gly) to amino acid number 187 (Asn); and
(f) the amino acid sequence as shown in SEQ ID NO:18 from amino acid number 1 (Met) to amino acid number 187 (Asn).

23. An expression vector comprising the following operably linked elements:
a transcription promoter;
a DNA segment encoding a zpep14 polypeptide that is at least 90% identical to an amino acid sequence as shown in SEQ ID NO:18 from amino acid number 17 (Gly) to amino acid number 187 (Asn); and
a transcription terminator.

24. An expression vector according to claim 23, further comprising a secretory signal sequence operably linked to the DNA segment.

25. A cultured cell into which has been introduced an expression vector according to claim 23, wherein the cell expresses a polypeptide encoded by the DNA segment.

26. An isolated polypeptide comprising a sequence of amino acid residues that is at least 90% identical to an amino acid sequence selected from the group consisting of:
(a) the amino acid sequence as shown in SEQ ID NO:18 from amino acid number 17 (Gly) to amino acid number 91 (Cys);
(b) the amino acid sequence as shown in SEQ ID NO:18 from amino acid number 17 (Gly) to amino acid number 170 (Gly);
(c) the amino acid sequence as shown in SEQ ID NO:18 from amino acid number 95 (Asn) to amino acid number 170 (Gly);
(d) the amino acid sequence as shown in SEQ ID NO:18 from amino acid number 173 (Ile) to amino acid number 187 (Asn);
(e) the amino acid sequence as shown in SEQ ID NO:18 from amino acid number 17 (Gly) to amino acid number 187 (Asn); and
(f) the amino acid sequence as shown in SEQ ID NO:18 from amino acid number 1 (Met) to amino acid number 187 (Asn),
wherein the amino acid percent identity is determined using a FASTA program with ktup=1, gap opening penalty=10, gap extension penalty=1, and substitution matrix=BLOSUM62, with other parameters set as default.

27. An isolated polypeptide according to claim 26, wherein the polypeptide comprises a sequence of amino acid residues that is selected from the group consisting of:
(a) the amino acid sequence as shown in SEQ ID NO:18 from amino acid number 17 (Gly) to amino acid number 91 (Cys);
(b) the amino acid sequence as shown in SEQ ID NO:18 from amino acid number 17 (Gly) to amino acid number 170 (Gly);
(c) the amino acid sequence as shown in SEQ ID NO:18 from amino acid number 95 (Asn) to amino acid number 170 (Gly);
(d) the amino acid sequence as shown in SEQ ID NO:18 from amino acid number 173 (Ile) to amino acid number 187 (Asn);
(e) the amino acid sequence as shown in SEQ ID NO:18 from amino acid number 17 (Gly) to amino acid number 187 (Asn); and
(f) the amino acid sequence as shown in SEQ ID NO:18 from amino acid number 1 (Met) to amino acid number 187 (Asn).

28. A method of producing a zpep14 polypeptide comprising:
culturing a cell according to claim 25; and
isolating the zpep14 polypeptide produced by the cell.

29. A method of producing an antibody to a zpep14 polypeptide comprising the following steps in order:
inoculating an animal with a polypeptide selected from the group consisting of:
(a) a polypeptide according to claim 26;
(b) a polypeptide consisting of the amino acid sequence of SEQ ID NO:18 from amino acid number 17 (Gly) to 91 (Cys);
(c) a polypeptide consisting of the amino acid sequence of SEQ ID NO:18 from amino acid number 17 (Gly) to 170 (Gly);
(d) a polypeptide consisting of the amino acid sequence of SEQ ID NO:18 from amino acid number 95 (Asn) to 170 (Gly);
(e) a polypeptide consisting of the amino acid sequence of SEQ ID NO:18 from amino acid number 173 (Ile) to 187 (Asn);
(f) a polypeptide consisting of the amino acid sequence of SEQ ID NO:18 from amino acid number 17 (Gly) to 187 (Asn); and
(g) a polypeptide consisting of a hydrophilic peptide predicted from a murine zpep14 hydrophobicity plot using a Hopp/Woods hydrophilicity profile based on a sliding six-residue window, with buried G, S, and T residues and exposed H, Y, and W residues ignored; and
and wherein the polypeptide elicits an immune response in the animal to produce the antibody; and isolating the antibody from the animal.

30. An antibody produced by the method of claim 29, which binds to a zpep14 polypeptide.

31. The antibody of claim 30, wherein the antibody is a monoclonal antibody.

32. An antibody which binds to a polypeptide of claim 26.
